SEQUENCE LISTING (39 sequences)

SEQ ID NO: 1 - DNA, 3384 bp, Artificial Sequence
  Made in Lab - Expression cassette
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcgggatccgccaccatgggctccatgtttcggagcgaggaggtggccctggtccagctctttctgcccacagcggctgcctacacctgcgtgagtcggctgggcgagctgggcctcgtggagttcagagacctcaacgcctcggtgagcgccttccagagacgctttgtggttgatgttcggcgctgtgaggagctggagaagaccttcaccttcctgcaggaggaggtgcggcgggctgggctggtcctgcccccgccaaaggggaggctgccggcacccccaccccgggacctgctgcgcatccaggaggagacggagcgcctggcccaggagctgcgggatgtgcggggcaaccagcaggccctgcgggcccagctgcaccagctgcagctccacgccgccgtgctacgccagggccatgaacctcagctggcagccgcccacacagatggggcctcagagaggacgcccctgctccaggcccccggggggccgcaccaggacctgagggtcaactttgtggcaggtgccgtggagccccacaaggcccctgccctagagcgcctgctctggagggcctgcagaggcttcctcattgccagcttcagggagctggagcagccgctggagcaccccgtgacgggcgagccagccacgtggatgaccttcctcatctcctactggggtgagcagatcggacagaagatccgcaagatcacggactgcttccactgccacgtcttcccgtttctgcagcaggaggaggcccgcctcggggccctgcagcagctgcaacagcagagccaggagctgcaggaggtcctcggggagacagagcggttcctgagccaggtgctaggccgggtgctgcagctgctgccgccagggcaggtgcaggtccacaagatgaaggccgtgtacctggccctgaaccagtgcagcgtgagcaccacgcacaagtgcctcattgccgaggcctggtgctctgtgcgagacctgcccgccctgcaggaggccctgcgggacagctcgatggaggagggagtgagtgccgtggctcaccgcatcccctgccgggacatgccccccacactcatccgcaccaaccgcttcacggccagcttccagggcatcgtggatgcctacggcgtgggccgctaccaggaggtcaaccccgctccctacaccatcatcaccttccccttcctgtttgctgtgatgttcggggatgtgggccacgggctgctcatgttcctcttcgccctggccatggtccttgcggagaaccgaccggctgtgaaggccgcgcagaacgagatctggcagactttcttcaggggccgctacctgctcctgcttatgggcctgttctccatctacaccggcttcatctacaacgagtgcttcagtcgcgccaccagcatcttcccctcgggctggagtgtggccgccatggccaaccagtctggctggagtgatgcattcctggcccagcacacgatgcttaccctggaccccaacgtcaccggtgtcttcctgggaccctacccctttggcatcgatcctatttggagcctggctgccaaccacttgagcttcctcaactccttcaagatgaagatgtccgtcatcctgggcgtcgtgcacatggcctttggggtggtcctcggagtcttcaaccacgtgcactttggccagaggcaccggctgctgctggagacgctgccggagctcaccttcctgctgggactcttcggttacctcgtgttcctagtcatctacaagtggctgtgtgtctgggctgccagggccgcctcggcccccagcatcctcatccacttcatcaacatgttcctcttctcccacagccccagcaacaggctgctctacccccggcaggaggtggtccaggccacgctggtggtcctggccttggccatggtgcccatcctgctgcttggcacacccctgcacctgctgcaccgccaccgccgccgcctgcggaggaggcccgctgaccgacaggaggaaaacaaggccgggttgctggacctgcctgacgcatctgtgaatggctggagctccgatgaggaaaaggcagggggcctggatgatgaagaggaggccgagctcgtcccctccgaggtgctcatgcaccaggccatccacaccatcgagttctgcctgggctgcgtctccaacaccgcctcctacctgcgcctgtgggccctgagcctggcccacgcccagctgtccgaggttctgtgggccatggtgatgcgcataggcctgggcctgggccgggaggtgggcgtggcggctgtggtgctggtccccatctttgccgcctttgccgtgatgaccgtggctatcctgctggtgatggagggactctcagccttcctgcacgccctgcggctgcactgggtggaattccagaacaagttctactcaggcacgggctacaagctgagtcccttcaccttcgctgccacagatgactagtaagtcgacggatcccccgggctgcaggaattcgagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttcg

SEQ ID NO: 2 - DNA, 243 bp, Homo sapiens
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgc

SEQ ID NO: 3 - DNA, 2493 bp, Homo sapiens
atgggctccatgtttcggagcgaggaggtggccctggtccagctctttctgcccacagcggctgcctacacctgcgtgagtcggctgggcgagctgggcctcgtggagttcagagacctcaacgcctcggtgagcgccttccagagacgctttgtggttgatgttcggcgctgtgaggagctggagaagaccttcaccttcctgcaggaggaggtgcggcgggctgggctggtcctgcccccgccaaaggggaggctgccggcacccccaccccgggacctgctgcgcatccaggaggagacggagcgcctggcccaggagctgcgggatgtgcggggcaaccagcaggccctgcgggcccagctgcaccagctgcagctccacgccgccgtgctacgccagggccatgaacctcagctggcagccgcccacacagatggggcctcagagaggacgcccctgctccaggcccccggggggccgcaccaggacctgagggtcaactttgtggcaggtgccgtggagccccacaaggcccctgccctagagcgcctgctctggagggcctgcagaggcttcctcattgccagcttcagggagctggagcagccgctggagcaccccgtgacgggcgagccagccacgtggatgaccttcctcatctcctactggggtgagcagatcggacagaagatccgcaagatcacggactgcttccactgccacgtcttcccgtttctgcagcaggaggaggcccgcctcggggccctgcagcagctgcaacagcagagccaggagctgcaggaggtcctcggggagacagagcggttcctgagccaggtgctaggccgggtgctgcagctgctgccgccagggcaggtgcaggtccacaagatgaaggccgtgtacctggccctgaaccagtgcagcgtgagcaccacgcacaagtgcctcattgccgaggcctggtgctctgtgcgagacctgcccgccctgcaggaggccctgcgggacagctcgatggaggagggagtgagtgccgtggctcaccgcatcccctgccgggacatgccccccacactcatccgcaccaaccgcttcacggccagcttccagggcatcgtggatgcctacggcgtgggccgctaccaggaggtcaaccccgctccctacaccatcatcaccttccccttcctgtttgctgtgatgttcggggatgtgggccacgggctgctcatgttcctcttcgccctggccatggtccttgcggagaaccgaccggctgtgaaggccgcgcagaacgagatctggcagactttcttcaggggccgctacctgctcctgcttatgggcctgttctccatctacaccggcttcatctacaacgagtgcttcagtcgcgccaccagcatcttcccctcgggctggagtgtggccgccatggccaaccagtctggctggagtgatgcattcctggcccagcacacgatgcttaccctggaccccaacgtcaccggtgtcttcctgggaccctacccctttggcatcgatcctatttggagcctggctgccaaccacttgagcttcctcaactccttcaagatgaagatgtccgtcatcctgggcgtcgtgcacatggcctttggggtggtcctcggagtcttcaaccacgtgcactttggccagaggcaccggctgctgctggagacgctgccggagctcaccttcctgctgggactcttcggttacctcgtgttcctagtcatctacaagtggctgtgtgtctgggctgccagggccgcctcggcccccagcatcctcatccacttcatcaacatgttcctcttctcccacagccccagcaacaggctgctctacccccggcaggaggtggtccaggccacgctggtggtcctggccttggccatggtgcccatcctgctgcttggcacacccctgcacctgctgcaccgccaccgccgccgcctgcggaggaggcccgctgaccgacaggaggaaaacaaggccgggttgctggacctgcctgacgcatctgtgaatggctggagctccgatgaggaaaaggcagggggcctggatgatgaagaggaggccgagctcgtcccctccgaggtgctcatgcaccaggccatccacaccatcgagttctgcctgggctgcgtctccaacaccgcctcctacctgcgcctgtgggccctgagcctggcccacgcccagctgtccgaggttctgtgggccatggtgatgcgcataggcctgggcctgggccgggaggtgggcgtggcggctgtggtgctggtccccatctttgccgcctttgccgtgatgaccgtggctatcctgctggtgatggagggactctcagccttcctgcacgccctgcggctgcactgggtggaattccagaacaagttctactcaggcacgggctacaagctgagtcccttcaccttcgctgccacagatgactag

SEQ ID NO: 4 - DNA, 606 bp, Woodchuck hepatitis virus
attcgagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttcg

SEQ ID NO: 5 - protein, 830 aa, Homo sapiens
MGSMFRSEEVALVQLFLPTAAAYTCVSRLGELGLVEFRDLNASVSAFQRRFVVDVRRCEELEKTFTFLQEEVRRAGLVLPPPKGRLPAPPPRDLLRIQEETERLAQELRDVRGNQQALRAQLHQLQLHAAVLRQGHEPQLAAAHTDGASERTPLLQAPGGPHQDLRVNFVAGAVEPHKAPALERLLWRACRGFLIASFRELEQPLEHPVTGEPATWMTFLISYWGEQIGQKIRKITDCFHCHVFPFLQQEEARLGALQQLQQQSQELQEVLGETERFLSQVLGRVLQLLPPGQVQVHKMKAVYLALNQCSVSTTHKCLIAEAWCSVRDLPALQEALRDSSMEEGVSAVAHRIPCRDMPPTLIRTNRFTASFQGIVDAYGVGRYQEVNPAPYTIITFPFLFAVMFGDVGHGLLMFLFALAMVLAENRPAVKAAQNEIWQTFFRGRYLLLLMGLFSIYTGFIYNECFSRATSIFPSGWSVAAMANQSGWSDAFLAQHTMLTLDPNVTGVFLGPYPFGIDPIWSLAANHLSFLNSFKMKMSVILGVVHMAFGVVLGVFNHVHFGQRHRLLLETLPELTFLLGLFGYLVFLVIYKWLCVWAARAASAPSILIHFINMFLFSHSPSNRLLYPRQEVVQATLVVLALAMVPILLLGTPLHLLHRHRRRLRRRPADRQEENKAGLLDLPDASVNGWSSDEEKAGGLDDEEEAELVPSEVLMHQAIHTIEFCLGCVSNTASYLRLWALSLAHAQLSEVLWAMVMRIGLGLGREVGVAAVVLVPIFAAFAVMTVAILLVMEGLSAFLHALRLHWVEFQNKFYSGTGYKLSPFTFAATDD

SEQ ID NO: 6 - DNA, 13 bp, Artificial Sequence
  Consensus Kozak sequence
gccgccaccatgg

SEQ ID NO: 7 - DNA, 387 bp, Mus musculus
tggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 8-13 - skipped (6 entries)

SEQ ID NO: 14 - RNA, 13 nt, Artificial Sequence
  Consensus Kozak sequence
gccgccrccaugg

SEQ ID NO: 15 - RNA, 8 nt, Artificial Sequence
  Kozak sequence
  misc_feature (2)..(3): n is A, C, T, G or U
  misc_feature (4)..(4): n is a, c, g, or u
  misc_feature (8)..(8): n is A, C, T, G or U
agnnaugn

SEQ ID NO: 16 - RNA, 7 nt, Artificial Sequence
  Kozak sequence
  misc_feature (2)..(3): n is A, C, T, G or U
annaugg

SEQ ID NO: 17 - RNA, 7 nt, Artificial Sequence
  Kozak sequence
accaugg

SEQ ID NO: 18 - RNA, 10 nt, Artificial Sequence
  n is A, C, T, G or U
gacaccaugg

SEQ ID NO: 19 - DNA, 235 bp, Bos taurus
tcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaggacaatagcaggcatgctggggatgcggtgggctctatggcttctg

SEQ ID NO: 20 - DNA, 222 bp, simian virus 40
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggta

SEQ ID NO: 21 - DNA, 202 bp, Homo sapiens
ctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 22 - DNA, 141 bp, Escherichia coli
gtagaattggtaaagagagtcgtgtaaaatatcgagttcgcacatcttgttgtctgattattgatttttggcgaaaccatttgatcatatgacaagatgtgtatctaccttaacttaatgattttgataaaaatcattagg

SEQ ID NO: 23 - DNA, 7660 bp, Artificial Sequence
  Made in Lab - plasmid construct
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatcgattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcgggatccgccaccatgggctccatgtttcggagcgaggaggtggccctggtccagctctttctgcccacagcggctgcctacacctgcgtgagtcggctgggcgagctgggcctcgtggagttcagagacctcaacgcctcggtgagcgccttccagagacgctttgtggttgatgttcggcgctgtgaggagctggagaagaccttcaccttcctgcaggaggaggtgcggcgggctgggctggtcctgcccccgccaaaggggaggctgccggcacccccaccccgggacctgctgcgcatccaggaggagacggagcgcctggcccaggagctgcgggatgtgcggggcaaccagcaggccctgcgggcccagctgcaccagctgcagctccacgccgccgtgctacgccagggccatgaacctcagctggcagccgcccacacagatggggcctcagagaggacgcccctgctccaggcccccggggggccgcaccaggacctgagggtcaactttgtggcaggtgccgtggagccccacaaggcccctgccctagagcgcctgctctggagggcctgcagaggcttcctcattgccagcttcagggagctggagcagccgctggagcaccccgtgacgggcgagccagccacgtggatgaccttcctcatctcctactggggtgagcagatcggacagaagatccgcaagatcacggactgcttccactgccacgtcttcccgtttctgcagcaggaggaggcccgcctcggggccctgcagcagctgcaacagcagagccaggagctgcaggaggtcctcggggagacagagcggttcctgagccaggtgctaggccgggtgctgcagctgctgccgccagggcaggtgcaggtccacaagatgaaggccgtgtacctggccctgaaccagtgcagcgtgagcaccacgcacaagtgcctcattgccgaggcctggtgctctgtgcgagacctgcccgccctgcaggaggccctgcgggacagctcgatggaggagggagtgagtgccgtggctcaccgcatcccctgccgggacatgccccccacactcatccgcaccaaccgcttcacggccagcttccagggcatcgtggatgcctacggcgtgggccgctaccaggaggtcaaccccgctccctacaccatcatcaccttccccttcctgtttgctgtgatgttcggggatgtgggccacgggctgctcatgttcctcttcgccctggccatggtccttgcggagaaccgaccggctgtgaaggccgcgcagaacgagatctggcagactttcttcaggggccgctacctgctcctgcttatgggcctgttctccatctacaccggcttcatctacaacgagtgcttcagtcgcgccaccagcatcttcccctcgggctggagtgtggccgccatggccaaccagtctggctggagtgatgcattcctggcccagcacacgatgcttaccctggaccccaacgtcaccggtgtcttcctgggaccctacccctttggcatcgatcctatttggagcctggctgccaaccacttgagcttcctcaactccttcaagatgaagatgtccgtcatcctgggcgtcgtgcacatggcctttggggtggtcctcggagtcttcaaccacgtgcactttggccagaggcaccggctgctgctggagacgctgccggagctcaccttcctgctgggactcttcggttacctcgtgttcctagtcatctacaagtggctgtgtgtctgggctgccagggccgcctcggcccccagcatcctcatccacttcatcaacatgttcctcttctcccacagccccagcaacaggctgctctacccccggcaggaggtggtccaggccacgctggtggtcctggccttggccatggtgcccatcctgctgcttggcacacccctgcacctgctgcaccgccaccgccgccgcctgcggaggaggcccgctgaccgacaggaggaaaacaaggccgggttgctggacctgcctgacgcatctgtgaatggctggagctccgatgaggaaaaggcagggggcctggatgatgaagaggaggccgagctcgtcccctccgaggtgctcatgcaccaggccatccacaccatcgagttctgcctgggctgcgtctccaacaccgcctcctacctgcgcctgtgggccctgagcctggcccacgcccagctgtccgaggttctgtgggccatggtgatgcgcataggcctgggcctgggccgggaggtgggcgtggcggctgtggtgctggtccccatctttgccgcctttgccgtgatgaccgtggctatcctgctggtgatggagggactctcagccttcctgcacgccctgcggctgcactgggtggaattccagaacaagttctactcaggcacgggctacaagctgagtcccttcaccttcgctgccacagatgactagtaagtcgacggatcccccgggctgcaggaattcgagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttcgaattcgatatcaagctgtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggtagcccgcctaatgagcgggcttttttttcttaggccttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacctgtacatttatattggctcatgtccaacattaccgccatgttg

SEQ ID NO: 24 - DNA, 511 bp, Homo sapiens
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 25 - DNA, 7613 bp, Artificial Sequence
  Made in Lab - plasmid construct
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcgggatccgccaccatgggctccatgtttcggagcgaggaggtggccctggtccagctctttctgcccacagcggctgcctacacctgcgtgagtcggctgggcgagctgggcctcgtggagttcagagacctcaacgcctcggtgagcgccttccagagacgctttgtggttgatgttcggcgctgtgaggagctggagaagaccttcaccttcctgcaggaggaggtgcggcgggctgggctggtcctgcccccgccaaaggggaggctgccggcacccccaccccgggacctgctgcgcatccaggaggagacggagcgcctggcccaggagctgcgggatgtgcggggcaaccagcaggccctgcgggcccagctgcaccagctgcagctccacgccgccgtgctacgccagggccatgaacctcagctggcagccgcccacacagatggggcctcagagaggacgcccctgctccaggcccccggggggccgcaccaggacctgagggtcaactttgtggcaggtgccgtggagccccacaaggcccctgccctagagcgcctgctctggagggcctgcagaggcttcctcattgccagcttcagggagctggagcagccgctggagcaccccgtgacgggcgagccagccacgtggatgaccttcctcatctcctactggggtgagcagatcggacagaagatccgcaagatcacggactgcttccactgccacgtcttcccgtttctgcagcaggaggaggcccgcctcggggccctgcagcagctgcaacagcagagccaggagctgcaggaggtcctcggggagacagagcggttcctgagccaggtgctaggccgggtgctgcagctgctgccgccagggcaggtgcaggtccacaagatgaaggccgtgtacctggccctgaaccagtgcagcgtgagcaccacgcacaagtgcctcattgccgaggcctggtgctctgtgcgagacctgcccgccctgcaggaggccctgcgggacagctcgatggaggagggagtgagtgccgtggctcaccgcatcccctgccgggacatgccccccacactcatccgcaccaaccgcttcacggccagcttccagggcatcgtggatgcctacggcgtgggccgctaccaggaggtcaaccccgctccctacaccatcatcaccttccccttcctgtttgctgtgatgttcggggatgtgggccacgggctgctcatgttcctcttcgccctggccatggtccttgcggagaaccgaccggctgtgaaggccgcgcagaacgagatctggcagactttcttcaggggccgctacctgctcctgcttatgggcctgttctccatctacaccggcttcatctacaacgagtgcttcagtcgcgccaccagcatcttcccctcgggctggagtgtggccgccatggccaaccagtctggctggagtgatgcattcctggcccagcacacgatgcttaccctggaccccaacgtcaccggtgtcttcctgggaccctacccctttggcatcgatcctatttggagcctggctgccaaccacttgagcttcctcaactccttcaagatgaagatgtccgtcatcctgggcgtcgtgcacatggcctttggggtggtcctcggagtcttcaaccacgtgcactttggccagaggcaccggctgctgctggagacgctgccggagctcaccttcctgctgggactcttcggttacctcgtgttcctagtcatctacaagtggctgtgtgtctgggctgccagggccgcctcggcccccagcatcctcatccacttcatcaacatgttcctcttctcccacagccccagcaacaggctgctctacccccggcaggaggtggtccaggccacgctggtggtcctggccttggccatggtgcccatcctgctgcttggcacacccctgcacctgctgcaccgccaccgccgccgcctgcggaggaggcccgctgaccgacaggaggaaaacaaggccgggttgctggacctgcctgacgcatctgtgaatggctggagctccgatgaggaaaaggcagggggcctggatgatgaagaggaggccgagctcgtcccctccgaggtgctcatgcaccaggccatccacaccatcgagttctgcctgggctgcgtctccaacaccgcctcctacctgcgcctgtgggccctgagcctggcccacgcccagctgtccgaggttctgtgggccatggtgatgcgcataggcctgggcctgggccgggaggtgggcgtggcggctgtggtgctggtccccatctttgccgcctttgccgtgatgaccgtggctatcctgctggtgatggagggactctcagccttcctgcacgccctgcggctgcactgggtggaattccagaacaagttctactcaggcacgggctacaagctgagtcccttcaccttcgctgccacagatgactagtaagtcgacggatcccccgggctgcaggaattcgagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttcgaattcgatatcaagctgtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggtagcccgcctaatgagcgggcttttttttcttaggccttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacctgtacatttatattggctcatgtccaacattaccgccatgttg

SEQ ID NO: 26 - DNA, 47 bp, Artificial Sequence
  Made in Lab - section of plasmid construct
gatcacgagactagcctcgagaagcttgatcgattggctccggtgcc

SEQ ID NO: 27 - DNA, 7646 bp, Artificial Sequence
  Made in lab - plasmid construct
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcgggatccgccaccatgggctccatgtttcggagcgaggaggtggccctggtccagctctttctgcccacagcggctgcctacacctgcgtgagtcggctgggcgagctgggcctcgtggagttcagagacctcaacgcctcggtgagcgccttccagagacgctttgtggttgatgttcggcgctgtgaggagctggagaagaccttcaccttcctgcaggaggaggtgcggcgggctgggctggtcctgcccccgccaaaggggaggctgccggcacccccaccccgggacctgctgcgcatccaggaggagacggagcgcctggcccaggagctgcgggatgtgcggggcaaccagcaggccctgcgggcccagctgcaccagctgcagctccacgccgccgtgctacgccagggccatgaacctcagctggcagccgcccacacagatggggcctcagagaggacgcccctgctccaggcccccggggggccgcaccaggacctgagggtcaactttgtggcaggtgccgtggagccccacaaggcccctgccctagagcgcctgctctggagggcctgcagaggcttcctcattgccagcttcagggagctggagcagccgctggagcaccccgtgacgggcgagccagccacgtggatgaccttcctcatctcctactggggtgagcagatcggacagaagatccgcaagatcacggactgcttccactgccacgtcttcccgtttctgcagcaggaggaggcccgcctcggggccctgcagcagctgcaacagcagagccaggagctgcaggaggtcctcggggagacagagcggttcctgagccaggtgctaggccgggtgctgcagctgctgccgccagggcaggtgcaggtccacaagatgaaggccgtgtacctggccctgaaccagtgcagcgtgagcaccacgcacaagtgcctcattgccgaggcctggtgctctgtgcgagacctgcccgccctgcaggaggccctgcgggacagctcgatggaggagggagtgagtgccgtggctcaccgcatcccctgccgggacatgccccccacactcatccgcaccaaccgcttcacggccagcttccagggcatcgtggatgcctacggcgtgggccgctaccaggaggtcaaccccgctccctacaccatcatcaccttccccttcctgtttgctgtgatgttcggggatgtgggccacgggctgctcatgttcctcttcgccctggccatggtccttgcggagaaccgaccggctgtgaaggccgcgcagaacgagatctggcagactttcttcaggggccgctacctgctcctgcttatgggcctgttctccatctacaccggcttcatctacaacgagtgcttcagtcgcgccaccagcatcttcccctcgggctggagtgtggccgccatggccaaccagtctggctggagtgatgcattcctggcccagcacacgatgcttaccctggaccccaacgtcaccggtgtcttcctgggaccctacccctttggcatcgatcctatttggagcctggctgccaaccacttgagcttcctcaactccttcaagatgaagatgtccgtcatcctgggcgtcgtgcacatggcctttggggtggtcctcggagtcttcaaccacgtgcactttggccagaggcaccggctgctgctggagacgctgccggagctcaccttcctgctgggactcttcggttacctcgtgttcctagtcatctacaagtggctgtgtgtctgggctgccagggccgcctcggcccccagcatcctcatccacttcatcaacatgttcctcttctcccacagccccagcaacaggctgctctacccccggcaggaggtggtccaggccacgctggtggtcctggccttggccatggtgcccatcctgctgcttggcacacccctgcacctgctgcaccgccaccgccgccgcctgcggaggaggcccgctgaccgacaggaggaaaacaaggccgggttgctggacctgcctgacgcatctgtgaatggctggagctccgatgaggaaaaggcagggggcctggatgatgaagaggaggccgagctcgtcccctccgaggtgctcatgcaccaggccatccacaccatcgagttctgcctgggctgcgtctccaacaccgcctcctacctgcgcctgtgggccctgagcctggcccacgcccagctgtccgaggttctgtgggccatggtgatgcgcataggcctgggcctgggccgggaggtgggcgtggcggctgtggtgctggtccccatctttgccgcctttgccgtgatgaccgtggctatcctgctggtgatggagggactctcagccttcctgcacgccctgcggctgcactgggtggaattccagaacaagttctactcaggcacgggctacaagctgagtcccttcaccttcgctgccacagatgactagtaagtcgacggatcccccgggctgcaggaattcgagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttcgaattcgatatcaagctgtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggtagcccgcctaatgagcgggcttttttttcttaggccttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacctgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgag

SEQ ID NO: 28 - DNA, 234 bp, human immunodeficiency virus
tggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 29 - DNA, 132 bp, Simian virus 40
aacttgtttattgcagcttataatggttacaaataaagcaatagcatcacaaatttcacaaataaagcatttttttcactgcattctagttgtggtttgtccaaactcatcaatgtatcttatcatgtctgg

SEQ ID NO: 30 - DNA, 160 bp, Simian virus 40
tcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagg

SEQ ID NO: 31 - DNA, 1015 bp, Artificial Sequence
  Made in Lab
tcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 32 - DNA, 139 bp, Escherichia coli
gtagaattggtaaagagagtcgtgtaaaatatcgagttcgcacatcttgttgtctgattattgatttttggcgaaaccatttgatcatatgacaagatgtgtatctaccttaacttaatgattttgataaaaatcatta

SEQ ID NO: 33 - DNA, 577 bp, Human betaherpesvirus 5
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagc

SEQ ID NO: 34 - DNA, 188 bp, human immunodeficiency virus
gtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgccc

SEQ ID NO: 35 - DNA, 45 bp, Human immunodeficiency virus 1
tgagtacgccaaaaattttgactagcggaggctagaaggagagag

SEQ ID NO: 36 - DNA, 362 bp, human immunodeficiency virus
atgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgct

SEQ ID NO: 37 - DNA, 858 bp, Artificial Sequence
  Made in Lab - plasmid element
gatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatc

SEQ ID NO: 38 - DNA, 118 bp, human immunodeficiency virus
ttttaaaagaaaaggggggattggggggtacagtgcaggggaaagaatagtagacataatagcaacagacatacaaactaaagaattacaaaaacaaattacaaaaattcaaaatttt

SEQ ID NO: 39 - DNA, 3847 bp, Artificial Sequence
  Made in lab - plasmid backbone construct
aacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggtagcccgcctaatgagcgggcttttttttcttaggccttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacctgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaatttt

(19) United States
(12) Patent Application Publication
(10) Pub. No.: US 2022/0218844 A1
(43) Pub. Date: Jul. 14, 2022

(54) GENE THERAPY VECTORS FOR INFANTILE MALIGNANT OSTEOPETROSIS

(71) Applicant: Spacecraft Seven, LLC, Cranbury, NJ (US)

(72) Inventors: Brian BEARD, Cranbury, NJ (US); David RICKS, Cranbury, NJ (US); Raj PRABHAKAR, Cranbury, NJ (US)

(73) Assignee: Spacecraft Seven, LLC, Cranbury, NJ (US)

(21) Appl. No.: 17/612,134

(22) PCT Filed: May 22, 2020
(86) PCT No.: PCT/US2020/034394

§ 371 Date: Nov. 17, 2021

Related U.S. Application Data
(60) Provisional application No. 62/852,216, filed on May 23, 2019.

(51) Int. Cl. A61K 48/00; C07K 14/47; C12N 15/86

(52) U.S. Cl. CPC A61K 48/005; C12N 2740/16043; C12N 15/86; C07K 14/4702

(57) ABSTRACT

The present disclosure provides improved gene therapy vectors comprising a polynucleotide sequence encoding a TCIRG1 polypeptide or functional variant thereof, methods of use thereof, pharmaceutical compositions, and more. In particular, the disclosure provides lentiviral vectors for treatment of infantile malignant osteopetrosis (IMO).

### CROSS-REFERENCE TO RELATED APPLICATIONS

This application claims the priority benefit of U.S. Provisional Application Ser. No. 62/852,216, filed on May 23, 2019, the contents of this application are hereby incorporated by reference herein in their entirety.

### STATEMENT REGARDING THE SEQUENCE LISTING

The Sequence Listing associated with this application is provided in text format in lieu of a paper copy, and is hereby incorporated by reference into the specification. The name of the text file containing the Sequence Listing is ROPA_003_01WO_ST25.txt. The text file is about 62 KB, created on May 22, 2020, and is being submitted electronically via EFS-Web.

### FIELD

The disclosure relates generally to gene therapy for diseases associated with mutations in a T cell immune regulator 1, ATPase H+ transporting V0 subunit a3 gene (TCIRG1). In particular, the disclosure provides gene therapy vectors and plasmids comprising expression cassettes that encode TCIRG1 protein (TCIRG1).

### BACKGROUND

Infantile malignant osteopetrosis (IMO) is a rare, recessive disorder characterized by increased bone mass caused by dysfunctional osteoclasts. The disease is most often caused by mutations in T cell immune regulator 1, ATPase H+ transporting V0 subunit a3 (TCIRG1). TCIRG1 is involved in osteoclasts' capacity to resorb bone.

Osteoclast function can be restored by lentiviral vector-mediated expression of TCIRG1. Moscatelli et al. Bone 57:1-9 (2013). Further studies show that lentiviral-mediated expression of TCIRG1 is regulated in the same manner as the endogenous gene product despite being expressed by a lentiviral vector with a constitutive physiologic promoter. Thudium et al. Calcif Tissue Int. 99:638-648 (2016). In addition, they established that the natural TCIRG1 gene sequence leads to higher level of protein expression and functional rescue in osteoclasts than a codon-optimized cDNA of the gene, even though mRNA levels from the latter were considerably higher. Furthermore, the data show that only a low fraction of human pre-osteoclasts with a functional TCIRG1 is needed to significantly increase resorptive function in vitro, likely due to the fusion of resorbing and non-resorbing osteoclasts, in line with previous results from the oc/oc mouse model of osteopetrosis. From both an efficacy and a safety perspective, the findings are encouraging for the further development of gene therapy for osteopetrosis.

There remains a need in the art for gene therapy vectors for TCIRG1 and method of treatment using such vectors. Furthermore, there is a need for reliable methods of producing such gene therapy vectors. The present disclosure provides such gene therapy vectors, methods of manufacture thereof, methods of use thereof, pharmaceutical compositions, and more.

### SUMMARY OF THE INVENTION

The present disclosure provides improved gene therapy vectors comprising a polynucleotide sequence encoding a TCIRG1 polypeptide or functional variant thereof, methods of use thereof, pharmaceutical compositions, and more.

In one aspect, the disclosure provides a transfer plasmid comprising an expression cassette comprising a coding polynucleotide encoding an isoform of T cell immune regulator 1 (TCIRG1) or a functional variant thereof, and a promoter, wherein the polynucleotide is operatively linked to the promoter, and wherein the transfer plasmid comprises an RNA-OUT repressor and a CMV IE promoter.

In some embodiments, the RNA-OUT repressor shares at least 95% identity or at least 99% identity to SEQ ID NO: 32.

In some embodiments, the CMV IE promoter shares at least 95% identity or at least 99% identity to SEQ ID NO: 33.

In some embodiments, the transfer plasmid comprises a pCCL backbone

In some embodiments, the pCCL backbone comprises the RNA-OUT repressor.

In some embodiments, the transfer plasmid shares at least 95% identity to SEQ ID NO: 39.

In some embodiments, the transfer plasmid comprises SEQ ID NO: 39.

In some embodiments, the promoter is an EFS promoter.

In some embodiments, the EFS promoter shares at least 95% identity with SEQ ID NO: 2.

In some embodiments, the EFS promoter is SEQ ID NO: 2.

In some embodiments, the coding polynucleotide shares at least 95% identity with SEQ ID NO: 3.

In some embodiments, the coding polynucleotide shares at least 99% identity with SEQ ID NO: 3.

In some embodiments, the coding polynucleotide is SEQ ID NO: 3.

In some embodiments, the expression cassette comprises a Woodchuck Hepatitis Virus (WHP) Posttranscriptional Regulatory Element (WPRE).

In some embodiments, the WPRE is SEQ ID NO: 4.

In some embodiments, the expression cassette shares at least 95% identity with SEQ ID NO: 1.

In some embodiments, the expression cassette is flanked by a 5′ long terminal repeat (LTR) and a 3′ LTR.

In some embodiments, the 5′ LTR is SEQ ID NO: 34 and/or the 3′ LTR is SEQ ID NO: 28.

In some embodiments, expression cassette shares at least 95% identity to SEQ ID NO: 1.

In some embodiments, expression cassette is SEQ ID NO: 1.

In another aspect, the disclosure provides an expression cassette comprising a polynucleotide encoding an isoform of T cell immune regulator 1 (TCIRG1), or a functional variant thereof, and EFS promoter, wherein optionally the polynucleotide is operatively linked to the EFS promoter.

In some embodiments, the coding polynucleotide shares at least 95% identity with SEQ ID NO: 3. In some embodiments, the coding polynucleotide shares at least 99% identity with SEQ ID NO: 3. In some embodiments, the coding polynucleotide is SEQ ID NO: 3. In some embodiments, the EFS promoter shares at least 95% identity with SEQ ID NO: 2. In some embodiments, the EFS promoter is SEQ ID NO: 2.

In some embodiments, the expression cassette comprises a Woodchuck Hepatitis Virus (WHP) Posttranscriptional Regulatory Element (WPRE). In some embodiments, the WPRE is SEQ ID NO: 4.

In some embodiments, the expression cassette shares at least 95% identity with SEQ ID NO: 1. In some embodiments, the expression cassette is SEQ ID NO: 1.

In another aspect, the disclosure provides a recombinant lentiviral genome, comprising in 5′ to 3′ order a lentiviral 5′ long terminal repeat (LTR); an expression cassette disclosed herein; and a lentiviral 3′LTR, wherein the recombinant lentiviral genome is replication incompetent.

In another aspect, the disclosure provides a lentiviral particle, comprising such a recombinant lentiviral genome.

In another aspect, the disclosure provides a transfer plasmid comprising such a recombinant lentiviral genome. In certain embodiments, the transfer plasmid comprises an RNA-OUT sequence. In some embodiments, the RNA-OUT sequence is SEQ ID NO: 22. In some embodiments, the RNA-OUT sequence is configured such that the transfer plasmid is capable of stable propagation in a packaging cell line.

In particular embodiments, the transfer plasmid does not comprise an antibiotic resistance gene or does not comprise an ampicillin resistance gene, such as AmpR.

In particular embodiments, the transfer plasmid comprises the sequence set forth in SEQ ID NO: 23.

In another aspect, the disclosure provides a method of generating a lentiviral particle, comprising transfecting a packaging cell line with any transfer plasmid of the disclosure, and optionally one or more additional plasmid, and culturing said packaging cell line. In some embodiments, the transfer plasmid is stably propagated in a bacterial host at 30-37° C. using shake flasks or fermentation for at least 1, 2, 3, 4, 5, 6, or 7 days.

In a related aspect, the disclosure provides a lentiviral particle produced using a transfer plasmid disclosed herein.

In another aspect, the disclosure provides a pharmaceutical composition comprising any lentiviral particle of the disclosure

In another aspect, the disclosure provides a modified cell comprising any expression cassette of the disclosure.

In another aspect, the disclosure provides a modified cell comprising any recombinant lentiviral genome of the disclosure.

In some embodiments, the modified cell lacks an endogenous functional TCIRG1 gene.

In some embodiments, the modified cell is derived from a subject having or suspected of having infantile malignant osteopetrosis (IMO).

In some embodiments, the modified cell expresses TCIRG1 or a functional variant thereof at a level similar to the level of expression of TCIRG1 observed in an osteoclast having a functional TCIRG1 gene.

In some embodiments, the modified cell expresses TCIRG1 or a functional variant thereof at a level similar to the level of expression of TCIRG1 observed in an osteoclast derived from a subject not having or suspected of having IMO.

In some embodiments, the modified cell is a hematopoietic stem cell (HSC).

In some embodiments, the modified cell is a CD34+ progenitor cell.

In some embodiments, the modified cell is derived a HSC isolated from a subject having or suspected of having IMO by apheresis.

In some embodiments, the modified cell is derived a HSC isolated from a subject having or suspected of having IMO by apheresis after mobilization of HSCs by administration of G-CSF, plerifaxor, or a combination of G-CSF and plerifaxor.

In some embodiments, the modified cell is derived from a population of cells enriched for CD34+ cells by magnetic capture.

In another aspect, the disclosure provides a pharmaceutical composition comprising any modified cell of the disclosure.

In another aspect, the disclosure provides an in vitro method of modifying one or more cells of a subject having or suspected of having IMO, comprising providing peripheral blood mononuclear cells (PBMCs) mobilized from the subject by administering to the subject a composition comprising G-CSF, plerifaxor, or a combination of G-CSF and plerifaxor; enriching the PBMCs for CD34+ cells by magnetic separation to generate a population of CD34-enriched cells; and contacting the CD34-enriched cells with a lentiviral particle comprising a recombinant lentiviral genome, comprising in 5′ to 3′ order: a lentiviral 5′ long terminal repeat (LTR); any expression cassette of the disclosure; and a lentiviral 3′LTR, wherein the recombinant lentiviral genome is replication incompetent.

In another aspect, the disclosure provides a method of treating infantile malignant osteopetrosis (IMO) in a subject having or suspected of having IMO, comprising administering any modified cell of the disclosure or any pharmaceutical composition of the disclosure to the subject.

In some embodiments, the method repopulates the HSC niche with modified cells expressing TCIRG1 or a functional variant thereof.

In some embodiments, the method repopulates the osteoclast niche with modified cells expressing TCIRG1 or a functional variant thereof.

In some embodiments, the method treats, ameliorates, prevents, reduces, inhibits, or relieves IMO.

In some embodiments, the method extends the mean overall survival of treated subjects by at least 1, 2, 3, 4, 5, 6, 7, 8, 9, 10 or more years.

In some embodiments, the method prevents the death of the subject from IMO.

In some embodiments, the subject is a human.

In some embodiments, the subject exhibited symptoms of IMO before treatment.

In some embodiments, the subject was identified as having reduced or non-detectable expression of TCIRG1 before treatment.

In some embodiments, the subject was identified as having a mutated TCIRG1 gene.

In some embodiments, the subject is an infant.

In some embodiments, the method comprises autologous treatment.

In some embodiments, the administration is performed via a intravenous infusion.

In another aspect, the disclosure provides a recombinant lentiviral genome for use in the preparation of a medicament for treating or preventing infantile malignant osteopetrosis (IMO), wherein the lentiviral genome comprises in 5′ to 3′ order a lentiviral 5′ long terminal repeat (LTR), any expression cassette of the disclosure, and a lentiviral 3′LTR; and wherein the recombinant lentiviral genome is replication incompetent.

In another aspect, the disclosure provides a lentiviral particle for use in the preparation of a medicament for treating or preventing infantile malignant osteopetrosis (IMO), comprising a recombinant lentiviral genome, wherein the lentiviral genome comprises in 5′ to 3′ order: a lentiviral 5′ long terminal repeat (LTR), any expression cassette of the disclosure, and a lentiviral 3′LTR; and wherein the recombinant lentiviral genome is replication incompetent.

Other features and advantages of the invention will be apparent from and encompassed by the following detailed description and claims.

### BRIEF DESCRIPTION OF THE DRAWINGS

FIG. 1 provides a diagram of a transfer plasmid for producing a lentiviral gene therapy vector encoding TCIRG1 (pCCL.PPT.EFS.tcirg1h.wpre).

FIG. 2 shows the gene sequence of an expression cassette (SEQ ID NO: 1), including in 5′ to 3′ order an elongation factor 1-α short (EFS) promoter (underlined; SEQ ID NO: 2), a polynucleotide encoding TCIRG1 (white letters on black; SEQ ID NO: 3), and a Woodchuck Hepatitis Virus (WHP) Posttranscriptional Regulatory Element (WPRE) (underlined and bold; SEQ ID NO: 4).

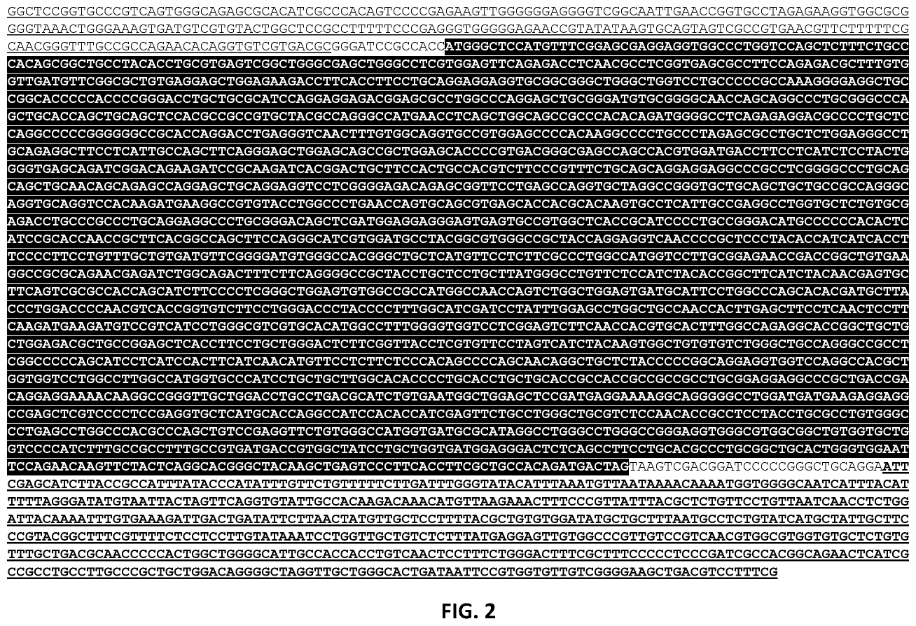

FIGS. 3A-3B provides a comparison of the stability of two different lentiviral plasmids. FIG. 3A shows a photograph of an agarose gel stained with ethidium bromide showing plasmid pRRL.PPT.EFS.tcirg1h.wpre, either not digested with a restriction enzyme (“Uncut”) or digested with AflIII (“AflIII”) or AflIII and NarI (“AflIII/NarI”). FIG. 3B shows a photograph of an agarose gel stained with ethidium bromide showing the plasmid pCCL.PPT.EFS.tcirg1h.wpre, either not digested with a restriction enzyme (“Uncut”) or digested with AflIII (“AflIII”) or AflIII and NarI (“AflIII/NarI”). FIG. 3C shows schematic diagrams of the pRRL.PPT.EFS.tcirg1h.wpre and pCCL.PPT.EFS.tcirg1h.wpre plasmids.

FIG. 4 depicts an illustrative process for lentiviral particle manufacturing.

FIGS. 5A-5B show (FIG. 5A) vector copy number (VCN) in bulk CD34+ cells liquid culture 6 and (FIG. 5B) 12 days after transduction. VCN was assessed by qPCR of extracted gDNA after culturing transduced CD34+ cells in SCGM complete media. VCN for each donor and the mean is represented for each transduction condition.

### DETAILED DESCRIPTION

The present inventors have shown that transplantation of autologous cells transduced with a lentiviral vector encoding TCIRG1 is effective in treating infantile malignant osteopetrosis (IMO). In addition, the inclusion of specific sequence elements in the expression cassette sequences of gene therapy vectors encoding TCIRG1 result in a safe and effective gene therapy for IMO. The present disclosure provides lentiviral vectors and plasmids encoding TCIRG1, including stable transfer plasmids advantageous for producing the lentiviral vectors.

### Vectors and Plasmids

The inventors have surprisingly discovered that production of lentivirus vector for TCIRG1 gene therapy at large scale is improved by modifying a pRRL plasmid containing the desired expression cassette in two ways: (i) replacing the pRRL vector backbone with a pCCL vector backbone, and (ii) replacing a conventional antibiotic resistance cassette in the pCCL backbone with the RNA-OUT selectable marker. The improved plasmid is then transfected into a lentiviral particle production system, along with helper plasmids, to produce the desired lentiviral vector.

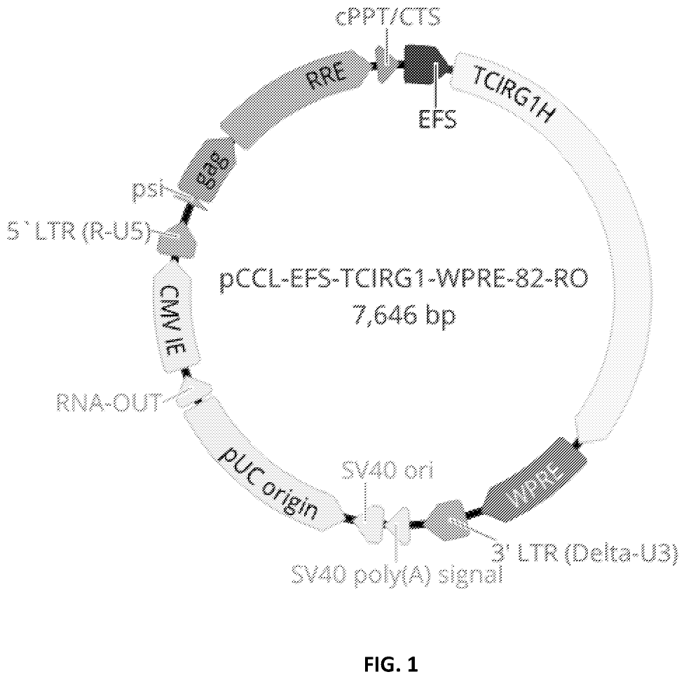

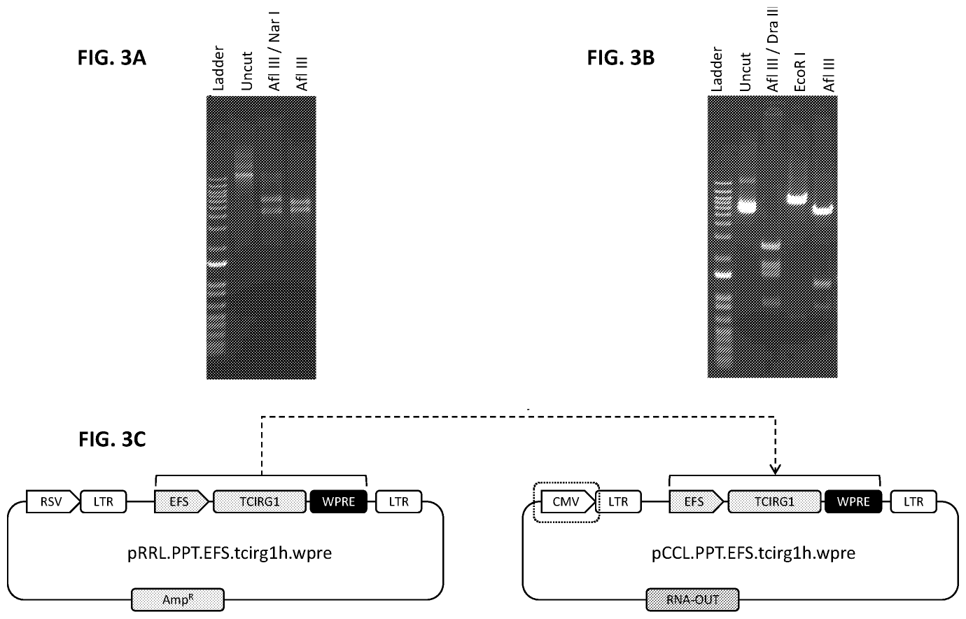

The resulting pCCL/RNA-OUT vector for TCIRG1 gene therapy (e.g., the vector depicted FIG. 1) has improved stability, reflected in higher plasmid yields from E. coli-based plasmid production and reduced levels of undesirable recombination products in the purified plasmid (shown in Example 1 and FIG. 3A-3C). This improvement to the transfer plasmid enables manufacture of lentiviral particles comprising the TCIRG1 expression cassette in yields sufficient for clinical testing and use. Further data provided herein demonstrate that lentiviral particles produced using the methods and compositions disclosed herein transduces CD34⁺ cells efficiently enough to reach clinically relevant vector copy number (VCN) levels.

In some embodiments, the disclosure provides a transfer plasmid that is a lentiviral vector based on the pCCL transfer plasmid used in third-generation lentiviral vector systems. The pCCL transfer plasmid contains the chimeric cytomegalovirus (CMV)-HIV 5′ LTR and vector backbones in which the simian virus 40 polyadenylation and (enhancerless) origin of replication sequences have been included downstream of the HIV 3′ LTR, replacing most of the human sequence remaining from the HIV integration site. The CCL 5′ hybrid long terminal repeat (LTR) is the enhancer and promoter (nucleotides −673 to −1 relative to the transcriptional start site; GenBank accession no. K03104) of cytomegalovirus (CMV) joined to the R region of HIV-1 LTR. In some embodiments, the transfer plasmid comprises an EFS promoter linked to the TCIRG1 gene with upstream RRE and cPPT/CTS elements and a downstream WPRE element (FIG. 1). In some embodiments, the transfer plasmid comprises a PGK promoter (SEQ ID NO: 24) linked to the TCIRG1 gene with upstream RRE and cPPT/CTS elements and a downstream WPRE element. In some embodiments, the transfer plasmid comprises an RNA-OUT element. Advantageously, the RNA-OUT sequence contributes to stable propagation of the transfer plasmid in a packing cell line. In some embodiments, the transfer plasmid does not comprise an antibiotic resistance gene, e.g., AmpR.

In some embodiments, the PGK promoter comprises a polynucleotide that shares at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity with SEQ ID NO: 24. In some embodiments, the PGK promoter comprises a polynucleotide that shares at least 80%, 85%, 90%, 95%, 99%, or 100% identity with SEQ ID NO: 24. In some embodiments, the PGK promoter has the sequence SEQ ID NO: 24.

(SEQ ID NO: 24)
GGGGTTGGGGTTGCGCCTTTTCCAAGGCAGCCCTG
GGTTTGCGCAGGGACGCGGCTGCTCTGGGCGTGGT
TCCGGGAAACGCAGCGGCGCCGACCCTGGGTCTCG
CACATTCTTCACGTCCGTTCGCAGCGTCACCCGGA
TCTTCGCCGCTACCCTTGTGGGCCCCCCGGCGACG
CTTCCTGCTCCGCCCCTAAGTCGGGAAGGTTCCTT
GCGGTTCGCGGCGTGCCGGACGTGACAAACGGAAG
CCGCACGTCTCACTAGTACCCTCGCAGACGGACAG
CGCCAGGGAGCAATGGCAGCGCGCCGACCGCGATG
GGCTGTGGCCAATAGCGGCTGCTCAGCAGGGCGCG
CCGAGAGCAGCGGCCGGGAAGGGGCGGTGCGGGAG
GCGGGGTGTGGGGCGGTAGTGTGGGCCCTGTTCCT
GCCCGCGCGGTGTTCCGCATTCTGCAAGCCTCCGG
AGCGCACGTCGGCAGTCGGCTCCCTCGTTGACCGA
ATCACCGACCTCTCTCCCCAG

In certain embodiments, the transfer plasmid is more stable than another plasmid comprising the same expression cassette when cultured or propagated in E. coli, thus resulting in a higher yield of plasmid, which is advantageous for use in producing vector. In some embodiments, the transfer plasmid is more stable that the pRRL.PPT.EFS.tcirg1h.wpre transfer plasmid. In particular embodiments, at least 2-fold, at least 5-fold, or at least 10-fold more of the transfer plasmid is produced as compared to the amount of pRRL.PPT.EFS.tcirg1h.wpre transfer plasmid produced under the same culture conditions.

In particular embodiments, the transfer plasmid is pCCL.PPT.EFS.tcirg1h.wpre or functional variants thereof, e.g., those disclosed herein. The disclosure provides in particular embodiments, the transfer plasmid pCCL.PPT.EFS.tcirg1h.wrpe or functional variants thereof. The transfer plasmid pCCL.PPT.EFS.tcirg1h.wrpe may have the sequence SEQ ID NO: 23.

Alternatively, the transfer plasmid pCCL.PPT.EFS.tcirg1h.wrpe may have the sequence SEQ ID NO: 25, in which the sequence GATCACGAGACTAGCCTCGAGAAGCTTGATCGATTGGCTCCGGTGCC (SEQ ID NO: 26) is deleted.

The sequence SEQ ID NO: 25 represents a circular plasmid. The same sequence permuted to start with the EFS promoter at base pair 1 is provided as SEQ ID NO: 27.

In some embodiments, the transfer plasmid comprises a polynucleotide that shares at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity with SEQ ID NO: 23. In some embodiments, the transfer plasmid comprises a polynucleotide that shares at least 80%, 85%, 90%, 95%, 99%, or 100% identity with SEQ ID NO: 23. In some embodiments, the transfer plasmid has the sequence SEQ ID NO: 23.

In some embodiments, the transfer plasmid comprises a polynucleotide that shares at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity with SEQ ID NO: 25. In some embodiments, the transfer plasmid comprises a polynucleotide that shares at least 80%, 85%, 90%, 95%, 99%, or 100% identity with SEQ ID NO: 25. In some embodiments, the transfer plasmid has the sequence SEQ ID NO: 25.

In some embodiments, the transfer plasmid comprises a polynucleotide that shares at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity with SEQ ID NO: 27. In some embodiments, the transfer plasmid comprises a polynucleotide that shares at least 80%, 85%, 90%, 95%, 99%, or 100% identity with SEQ ID NO: 27. In some embodiments, the transfer plasmid has the sequence SEQ ID NO: 27.

In some embodiments, the transfer plasmid is comprises one or more of the vector elements listed in Table 1.

TABLE 1
pCCL.PPT.EFS.tcirg1h.wpre Vector Elements

| | | Min. - Max. | | |
|---|---|---|---|---|
| | | Position in | | |
| | | Reference | Length | |
| | | Sequence | (base | |
| Name | Type | (nucleotide) | pairs) | SEQ ID NO: |
| EFS | Promoter | 1-243 | 243 | SEQ ID NO: 2 |
| TCIRG1 | CDS | 257-2,749 | 2,493 | SEQ ID NO: 3 |
| WPRE | Regulatory | 2,782-3,384 | 603 | SEQ ID NO: 4 |
| 3′LTR | LTR | 3,471-3,704 | 234 | SEQ ID NO: 28 |
| SV40 poly(A) | polyA signal | 3,776-3,907 | 132 | SEQ ID NO: 29 |
| SV40 ori | Origin of | 3,917-4,076 | 160 | SEQ ID NO: 30 |
| | replication | | | |
| pUC origin | Origin of | 4,115-5,129 | 1,015 | SEQ ID NO: 31 |
| | replication | | | |
| RNA-OUT | Repressor | 5,146-5,284 | 139 | SEQ ID NO: 32 |
| CMV IE | Promoter | 5,334-5,910 | 577 | SEQ ID NO: 33 |
| 5-LTR | LTR | 5,933-6,120 | 188 | SEQ ID NO: 34 |
| psi | Packaging | 6,222-6,266 | 45 | SEQ ID NO: 35 |
| gag | CDS | 6,267-6,628 | 362 | SEQ ID NO: 36 |
| RRE | Regulatory | 6,629-7,486 | 858 | SEQ ID NO: 37 |
| cPPT/CTS | Poly purine tract | 7,505-7,622 | 118 | SEQ ID NO: 38 |
| — | Backbone | 3,776-7,622 | 3,847 | SEQ ID NO: 39 |

In some embodiments, lentiviral particles are generated by transient transfection of a third-generation lentiviral vector system that includes pCCL.PPT.EFS.tcirg1h.wpre.

In some embodiments, the expression cassette comprises a polynucleotide that shares at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity with SEQ ID NO: 1. In some embodiments, the expression cassette comprises a polynucleotide that shares at least 80%, 85%, 90%, 95%, 99%, or 100% identity with SEQ ID NO: 1. In some embodiments, the expression cassette has the sequence SEQ ID NO: 1.

In some embodiments, the expression cassette comprises, in 5′ to 3′ order, an EFS promoter, a polynucleotide encoding T cell immune regulator 1, ATPase H+ transporting V0 subunit a3 (TCIRG1) or a functional variant thereof, and a Woodchuck Hepatitis Virus (WHP) Posttranscriptional Regulatory Element (WPRE). In some embodiments, the EFS promoter is operatively linked to the polynucleotide encoding an isoform of TCIRG1 or a functional variant thereof. Related embodiments comprise a transfer plasmid comprising the expression cassette, and a vector produced using the transfer plasmid.

In some embodiments, the EFS promoter comprises a polynucleotide that shares at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity with SEQ ID NO: 2. In some embodiments, the EFS promoter comprises a polynucleotide that shares at least 80%, 85%, 90%, 95%, 99%, or 100% identity with SEQ ID NO: 2. In some embodiments, the EFS promoter has the sequence SEQ ID NO: 2.

(SEQ ID NO: 2)
GGCTCCGGTGCCCGTCAGTGGGCAGAGCGCACATC
GCCCACAGTCCCCGAGAAGTTGGGGGGAGGGGTCG
GCAATTGAACCGGTGCCTAGAGAAGGTGGCGCGGG
GTAAACTGGGAAAGTGATGTCGTGTACTGGCTCCG
CCTTTTTCCCGAGGGTGGGGGAGAACCGTATATAA
GTGCAGTAGTCGCCGTGAACGTTCTTTTTCGCAAC
GGGTTTGCCGCCAGAACACAGGTGTCGTGACGC

In some embodiments, the polynucleotide encoding an isoform of TCIRG1 or a functional variant thereof comprises a polynucleotide that shares at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity with SEQ ID NO: 3. In some embodiments, the polynucleotide encoding an isoform of TCIRG1 or a functional variant thereof comprises a polynucleotide that shares at least 80%, 85%, 90%, 95%, 99%, or 100% identity with SEQ ID NO: 3. In some embodiments, the polynucleotide encoding an isoform of TCIRG1 or a functional variant thereof has the sequence SEQ ID NO: 3.

In some embodiments, the WPRE comprises a polynucleotide that shares at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity with SEQ ID NO: 4. In some embodiments, the WPRE comprises a polynucleotide that shares at least 80%, 85%, 90%, 95%, 99%, or 100% identity with SEQ ID NO: 4. In some embodiments, the WPRE has the sequence SEQ ID NO: 4.

(SEQ ID NO: 4)
ATTCGAGCATCTTACCGCCATTTATACCCATATTT
GTTCTGTTTTTCTTGATTTGGGTATACATTTAAAT
GTTAATAAAACAAAATGGTGGGGCAATCATTTACA
TTTTTAGGGATATGTAATTACTAGTTCAGGTGTAT
TGCCACAAGACAAACATGTTAAGAAACTTTCCCGT
TATTTACGCTCTGTTCCTGTTAATCAACCTCTGGA
TTACAAAATTTGTGAAAGATTGACTGATATTCTTA
ACTATGTTGCTCCTTTTACGCTGTGTGGATATGCT
GCTTTAATGCCTCTGTATCATGCTATTGCTTCCCG
TACGGCTTTCGTTTTCTCCTCCTTGTATAAATCCT
GGTTGCTGTCTCTTTATGAGGAGTTGTGGCCCGTT
GTCCGTCAACGTGGCGTGGTGTGCTCTGTGTTTGC
TGACGCAACCCCCACTGGCTGGGGCATTGCCACCA
CCTGTCAACTCCTTTCTGGGACTTTCGCTTTCCCC
CTCCCGATCGCCACGGCAGAACTCATCGCCGCCTG
CCTTGCCCGCTGCTGGACAGGGGCTAGGTTGCTGG
GCACTGATAATTCCGTGGTGTTGTCGGGGAAGCTG
ACGTCCTTTCG

In some embodiments, the isoform of TCIRG1 or a functional variant thereof comprises a polypeptide that shares at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity with SEQ ID NO: 5. In some embodiments, the isoform of TCIRG1 or a functional variant thereof comprises a polypeptide that shares at least 80%, 85%, 90%, 95%, 99%, or 100% identity with SEQ ID NO: 5. In some embodiments, the isoform of TCIRG1 or a functional variant thereof has the sequence SEQ ID NO: 5.

(SEQ ID NO: 5)
MGSMFRSEEVALVQLFLPTAAAYTCVSRLGELGLV
EFRDLNASVSAFQRRFVVDVRRCEELEKTFTFLQE
EVRRAGLVLPPPKGRLPAPPPRDLLRIQEETERLA
QELRDVRGNQQALRAQLHQLQLHAAVLRQGHEPQL
AAAHTDGASERTPLLQAPGGPHQDLRVNFVAGAVE
PHKAPALERLLWRACRGFLIASFRELEQPLEHPVT
GEPATWMTFLISYWGEQIGQKIRKITDCFHCHVFP
FLQQEEARLGALQQLQQQSQELQEVLGETERFLSQ
VLGRVLQLLPPGQVQVHKMKAVYLALNQCSVSTTH
KCLIAEAWCSVRDLPALQEALRDSSMEEGVSAVAH
RIPCRDMPPTLIRTNRFTASFQGIVDAYGVGRYQE
VNPAPYTIITFPFLFAVMFGDVGHGLLMFLFALAM
VLAENRPAVKAAQNEIWQTFFRGRYLLLLMGLFSI
YTGFIYNECFSRATSIFPSGWSVAAMANQSGWSDA
FLAQHTMLTLDPNVTGVFLGPYPFGIDPIWSLAAN
HLSFLNSFKMKMSVILGVVHMAFGVVLGVFNHVHF
GQRHRLLLETLPELTFLLGLFGYLVFLVIYKWLCV
WAARAASAPSILIHFINMFLFSHSPSNRLLYPRQE
VVQATLVVLALAMVPILLLGTPLHLLHRHRRRLRR
RPADRQEENKAGLLDLPDASVNGWSSDEEKAGGLD
DEEEAELVPSEVLMHQAIHTIEFCLGCVSNTASYL
RLWALSLAHAQLSEVLWAMVMRIGLGLGREVGVAA
VVLVPIFAAFAVMTVAILLVMEGLSAFLHALRLHW
VEFQNKFYSGTGYKLSPFTFAATDD

In an embodiment, the polynucleotide encoding an isoform of TCIRG1 or a functional variant thereof is codon-optimized for expression in a human host cell. In an embodiment, the polynucleotide encoding an isoform of TCIRG1 or a functional variant thereof is modified, or “codon optimized” to enhance expression by replacing infrequently represented codons with more frequently represented codons. In an embodiment, the polynucleotide encoding an isoform of TCIRG1 or a functional variant thereof is not codon-optimized. In an embodiment, the polynucleotide encoding an isoform of TCIRG1 or a functional variant thereof is not modified. In an embodiment, the polynucleotide encoding an isoform of TCIRG1 or a functional variant thereof is not codon-optimized. In an embodiment, the polynucleotide encoding an isoform of TCIRG1 or a functional variant thereof is a native polynucleotide sequence.

As used herein the term “transgene” refers to a polynucleotide encoding an isoform of TCIRG1 or a functional variant thereof.

The coding sequence is the portion of the mRNA sequence that encodes the amino acids for translation. During translation, each of 61 trinucleotide codons are translated to one of 20 amino acids, leading to a degeneracy, or redundancy, in the genetic code. However, different cell types, and different animal species, utilize tRNAs (each bearing an anticodon) coding for the same amino acids at different frequencies. When a gene sequence contains codons that are infrequently represented by the corresponding tRNA, the ribosome translation machinery may slow, impeding efficient translation. Expression can be improved via “codon optimization” for a particular species, where the coding sequence is altered to encode the same protein sequence, but utilizing codons that are highly represented, and/or utilized by highly expressed human proteins (Cid-Arregui et al., 2003; J. Virol. 77:4928).

In some embodiments, the coding sequence of the transgene is modified to replace codons infrequently expressed in mammal or in primates with codons frequently expressed in primates. For example, in some embodiments, the transgene encodes a polypeptide having at least 85% sequence identity to a reference polypeptide (e.g. wild-type TCIRG1; SEQ ID NO: 3)—for example, at least 90% sequence identity, at least 95% sequence identity, at least 98% identity, or at least 99% identity to the reference polypeptide—wherein at least one codon of the coding sequence has a higher tRNA frequency in humans than the corresponding codon in the sequence disclosed above or herein.

In an embodiment, the transgene comprises fewer alternative open reading frames than SEQ ID: 3. In an embodiment, the transgene is modified to enhance expression by termination or removal of open reading frames (ORFs) that do not encode the desired transgene. An open reading frame (ORF) is the nucleic acid sequence that follows a start codon and does not contain a stop codon. ORFs may be in the forward or reverse orientation, and may be “in frame” or “out of frame” compared with the gene of interest. Such open reading frames have the potential to be expressed in an expression cassette alongside the gene of interest, and could lead to undesired adverse effects. In some embodiments the transgene has been modified to remove open reading frames by further altering codon usage. This is done by eliminating one or more start codons (ATG) and/or introducing one or more stop codons (TAG, TAA, or TGA) in reverse orientation or out-of-frame to the desired ORF, while preserving the encoded amino acid sequence and, optionally, maintaining highly utilized codons in the gene of interest (i.e., avoiding codons with frequency <20%).

In variations of the present disclosure, the transgene coding sequence may be optimized by either of codon optimization and removal of non-transgene ORFs or using both techniques. In some cases, one removes or minimizes non-transgene ORFs after codon optimization in order to remove ORFs introduced during codon optimization.

In an embodiment, the transgene contains fewer CpG sites than SEQ ID: 3. Without being bound by theory, it is believed that the presence of CpG sites in a polynucleotide sequence is associated with the undesirable immunological responses of the host against a viral vector comprising the polynucleotide sequence. In some embodiments, the transgene is designed to reduce the number of CpG sites. Exemplary methods are provides in U.S. Patent Application Publication No. US20020065236A1.

In an embodiment, the transgene contains fewer cryptic splice sites than SEQ ID: 3. For the optimization, GeneArt® software may be used, e.g., to increase the GC content and/or remove cryptic splice sites in order to avoid transcriptional silencing and, therefore, increase transgene expression. Alternatively, any optimization method known in the art may be used. Removal of cryptic splice sites is described, for example, in International Patent Application Publication No. WO2004015106A1.

Also disclosed herein are expression cassettes and gene therapy vectors encoding TCIRG1, e.g., a TCIRG1 sequence disclosed herein, comprising: a consensus optimal Kozak sequence, a full-length polyadenylation (polyA) sequence (or substitution of full-length polyA for a truncated polyA), and minimal or no upstream (i.e. 5′) start codons (i.e. ATG sites).

In some embodiments, the expression cassette contains two or more of a 5′ long terminal repeat (LTR), an enhancer/promoter region, a consensus optimal Kozak sequence, a transgene (e.g., a transgene encoding a TCIRG1 disclosed herein), a 3′ untranslated region including a full-length polyA sequence, and a 3′ LTR.

In an embodiment, the expression cassette comprises a Kozak sequence operatively linked to the transgene. In an embodiment, the Kozak sequence is a consensus optimal Kozak sequence comprising or consisting of SEQ ID NO: 6.

(SEQ ID NO: 6)
GCCGCCACCATGG

In various embodiments, the expression cassette comprises an alternative Kozak sequence operatively linked to the transgene. In an embodiment, the Kozak sequence is an alternative Kozak sequence comprising or consisting of any one of SEQ ID NOs. 14-18.

(SEQ ID NO: 14)
(gcc)gccRccAUGG
(SEQ ID NO: 15)
AGNNAUGN
(SEQ ID NO: 16)
ANNAUGG
(SEQ ID NO: 17)
ACCAUGG
(SEQ ID NO: 18)
GACACCAUGG

In SEQ ID NO: 14, a lower-case letter denotes the most common base at a position where the base can nevertheless vary; an upper-case letter indicate a highly conserved base; indicates adenine or guanine. In SEQ ID NO: 14, the sequence in parentheses (gcc) is optional. IN SEQ ID NOs: 15-17, ‘N’ denotes any base.

A variety of sequences can be used in place of this consensus optimal Kozak sequence as the translation-initiation site and it is within the skill of those in the art to identify and test other sequences. See Kozak M. An analysis of vertebrate mRNA sequences: intimations of translational control. J. Cell Biol. 115 (4): 887-903 (1991).

In an embodiment, the expression cassette comprises a full-length polyA sequence operatively linked to the transgene. In an embodiment, the full-length polyA sequence comprises SEQ ID NO: 7.

(SEQ ID NO: 7)
TGGCTAATAAAGGAAATTTATTTTCATTGCAATAG
TGTGTTGGAATTTTTTGTGTCTCTCACTCGGAAGG
ACATATGGGAGGGCAAATCATTTAAAACATCAGAA
TGAGTATTTGGTTTAGAGTTTGGCAACATATGCCC
ATATGCTGGCTGCCATGAACAAAGGTTGGCTATAA
AGAGGTCATCAGTATATGAAACAGCCCCCTGCTGT
CCATTCCTTATTCCATAGAAAAGCCTTGACTTGAG
GTTAGATTTTTTTTATATTTTGTTTTGTGTTATTT
TTTTCTTTAACATCCCTAAAATTTTCCTTACATGT
TTTACTAGCCAGATTTTTCCTCCTCTCCTGACTAC
TCCCAGTCATAGCTGTCCCTCTTCTCTTATGGAGA
TC

Various alternative polyA sequences may be used in expression cassettes of the present disclosure, including without limitation, bovine growth hormone polyadenylation signal (bGHpA) (SEQ ID NO: 19), the SV40 early/late polyadenylation signal (SEQ ID NO: 20), and human growth hormone (HGH) polyadenylation signal (SEQ ID NO: 21).

(SEQ ID NO: 19)
TCGACTGTGCCTTCTAGTTGCCAGCCATCTGTTGT
TTGCCCCTCCCCCGTGCCTTCCTTGACCCTGGAAG
GTGCCACTCCCACTGTCCTTTCCTAATAAAATGAG
GAAATTGCATCGCATTGTCTGAGTAGGTGTCATTC
TATTCTGGGGGGTGGGGTGGGGCAGGACAGCAAGG
GGGAGGATTGGGAGGACAATAGCAGGCATGCTGGG
GATGCGGTGGGCTCTATGGCTTCTG
(SEQ ID NO: 20)
CAGACATGATAAGATACATTGATGAGTTTGGACAA
ACCACAACTAGAATGCAGTGAAAAAAATGCTTTAT
TTGTGAAATTTGTGATGCTATTGCTTTATTTGTAA
CCATTATAAGCTGCAATAAACAAGTTAACAACAAC
AATTGCATTCATTTTATGTTTCAGGTTCAGGGGGA
GATGTGGGAGGTTTTTTAAAGCAAGTAAAACCTCT
ACAAATGTGGTA
(SEQ ID NO: 21)
CTGCCCGGGTGGCATCCCTGTGACCCCTCCCCAGT
GCCTCTCCTGGCCCTGGAAGTTGCCACTCCAGTGC
CCACCAGCCTTGTCCTAATAAAATTAAGTTGCATC
ATTTTGTCTGACTAGGTGTCCTTCTATAATATTAT
GGGGTGGAGGGGGGTGGTATGGAGCAAGGGGCCCA
AGTTGGGAAGAAACCTGTAGGGCCTGC

In some embodiments, the expression cassette comprises an active fragment of a polyA sequence. In particular embodiments, the active fragment of the polA sequence comprises or consists of less than 20 base pair (bp), less than 50 bp, less than 100 bp, or less than 150 bp, e.g., of any of the polA sequences disclosed herein.

In some cases, expression of the transgene is increased by ensuring that the expression cassette does not contain competing ORFs. In an embodiment, the expression cassette comprises no start codon within 20, 30, 40, 50, 60, 70, 80, 90, 100, 200, 300, 400, or 500 basepairs 5′ of the start codon of the transgene. In an embodiment, the expression cassette comprises no start codon 5′ of the start codon of the transgene.

In an embodiment, the expression cassette comprises operatively linked, in the 5′ to 3′ direction, a first inverse terminal repeat, an enhancer/promoter region, introns, a consensus optimal Kozak sequence, the transgene, a 3′ untranslated region including a full-length polyA sequence, and a second inverse terminal repeat, where the expression cassette comprises no start codon 5′ to the start codon of the transgene.

In an embodiment, the enhancer/promoter region comprises, in the 5′ to 3′ direction: a CMV IE Enhancer and a Chicken Beta-Actin Promoter. In an embodiment, the enhancer/promoter region comprises a CAG promoter. As used herein “CAG promoter” refers to a polynucleotide sequence comprising a CMV early enhancer element, a chicken beta-actin promoter, the first exon and first intron of the chicken beta-actin gene, and a splice acceptor from the rabbit beta-globin gene.

In an embodiment, the enhancer/promoter region comprises an elongation factor 1α short promoter (EFS promoter) and is a shorter intron-less version of elongation factor 1α promoter. As used herein “EFS promoter” refers to a polynucleotide sequence comprising a short, intron-less form of EF1alpha. The EFS promoter has been recently used in many clinical trials. It is a cellular-derived enhancer/promoter with decreased cross-activation of nearby promoters, therefore hypothetically decreasing the risk of genotoxicity.

In an embodiment, the expression cassette shares at least 95% identity to a sequence selected from SEQ ID NOs: 1. In an embodiment, the expression cassette shares complete identity to a sequence selected from SEQ ID NOs: 1, or shares at least 80%, at least 85%, at least 90%, at least 95%, at least 98%, or at least 99% identity to a sequence selected from SEQ ID NOs: 1. In certain embodiments, the expression cassette comprises one or more modifications as compare to a sequence selected from SEQ ID NOs: 1. In particular embodiments, the one or more modifications comprises one or more of: removal of one or more (e.g., all) upstream ATG sequences, replacement of the Kozak sequence with an optimized consensus Kozak sequence or another Kozak sequence, including but not limited to any of those disclosed herein, and/or replacement of the polyadenylation sequence with a full-length polyadenylation sequence or another polyadenylation sequence, including but not limited to any of those disclosed herein. An illustrative configuration of genetic elements within these exemplary expression cassettes is depicted in FIG. 1.

In related embodiments, the disclosure provides gene therapy vectors comprising an expression cassette disclosed herein. Generally, the gene therapy vectors described herein comprise an expression cassette comprising a polynucleotide encoding one or more isoforms of TCIRG1, that allows for the expression of TCIRG1 to partially or wholly rectify deficient TCIRG1 protein expression levels and/or a defect in osteoclast formation in a subject in need thereof (e.g., a subject having Infantile Malignant Osteopetrosis or another disorder characterized by deficient osteoclast formation at least in part due to deficient TCIRG1 expression). In particular embodiments, the expression cassette comprises a polynucleotide sequence encoding TCIRG1 disclosed herein, e.g., SEQ ID NOs:3 or a sequence having at least 90%, at least 95%, at least 98%, or at least 99% identity to any of SEQ ID NO:s: 3. The gene therapy vectors can be viral or non-viral vectors. Illustrative non-viral vectors include, e.g., naked DNA, cationic liposome complexes, cationic polymer complexes, cationic liposome-polymer complexes, and exosomes. Examples of viral vector include, but are not limited, to adenoviral, retroviral, lentiviral, herpesvirus and adeno-associated virus (AAV) vectors.

Gene delivery viral vectors useful in the practice of the present invention can be constructed utilizing methodologies well known in the art of molecular biology. Typically, viral vectors carrying transgenes are assembled from polynucleotides encoding the transgene, suitable regulatory elements and elements necessary for production of viral proteins, which mediate cell transduction. Such recombinant viruses may be produced by techniques known in the art, e.g., by transfecting packaging cells or by transient transfection with helper plasmids or viruses. Typical examples of virus packaging cells include but are not limited to HeLa cells, SF9 cells (optionally with a baculovirus helper vector), 293 cells, etc. A Herpesvirus-based system can be used to produce AAV vectors, as described in US20170218395A1. Detailed protocols for producing such replication-defective recombinant viruses may be found for instance in W095/14785, W096/22378, U.S. Pat. Nos. 5,882,877, 6,013,516, 4,861,719, 5,278,056 and W094/19478, the complete contents of each of which is hereby incorporated by reference.

In some embodiments, the vector is a retroviral vector, or more specifically, a lentiviral vector. As used herein, the term “retrovirus” or “retroviral” refers an RNA virus that reverse transcribes its genomic RNA into a linear double-stranded DNA copy and subsequently covalently integrates its genomic DNA into a host genome. Retrovirus vectors are a common tool for gene delivery (Miller, 2000, Nature. 357: 455-460). Once the virus is integrated into the host genome, it is referred to as a “provirus.” The provirus serves as a template for RNA polymerase II and directs the expression of RNA molecules encoded by the virus.

Illustrative retroviruses (family Retroviridae) include, but are not limited to: (1) genus gammaretrovirus, such as, Moloney murine leukemia virus (M-MuLV), Moloney murine sarcoma virus (MoMSV), murine mammary tumor virus (MuMTV), gibbon ape leukemia virus (GaLV), and feline leukemia virus (FLV), (2) genus spumavirus, such as, simian foamy virus, (3) genus lentivirus, such as, human immunodeficiency virus-1 and simian immunodeficiency virus.

As used herein, the term “lentiviral” or “lentivirus” refers to a group (or genus) of complex retroviruses. Illustrative lentiviruses include, but are not limited to: HIV (human immunodeficiency virus; including HIV type 1, and HIV type 2; visna-maedi virus (VMV) virus; the caprine arthritis-encephalitis virus (CAEV); equine infectious anemia virus (EIAV); feline immunodeficiency virus (Hy); bovine immune deficiency virus (BIV); and simian immunodeficiency virus (SIV). In one embodiment, HIV-based vector backbones (i.e., HIV cis-acting sequence elements) are preferred.

Retroviral vectors, and more particularly, lentiviral vectors, may be used in practicing the present invention. Accordingly, the term “retroviral vector,” as used herein is meant to include “lentiviral vector”; and the term “retrovirus” as used herein is meant to include “lentivirus.”

The term viral vector may refer either to a vector or viral particle capable of transferring a nucleic acid into a cell or to the transferred nucleic acid itself. Viral vectors contain structural and/or functional genetic elements that are primarily derived from a virus. The term “retroviral vector” refers to a viral vector containing structural and functional genetic elements, or portions thereof, that are primarily derived from a retrovirus. The term “lentiviral vector” refers to a viral vector containing structural and functional genetic elements, or portions thereof, including LTRs that are primarily derived from a lentivirus. The term “hybrid” refers to a vector, LTR or other nucleic acid containing both retroviral, e.g., lentiviral, sequences and non-lentiviral viral sequences. In one embodiment, a hybrid vector refers to a vector or transfer plasmid comprising retroviral, e.g., lentiviral, sequences for reverse transcription, replication, integration and/or packaging.

In particular embodiments, the terms “lentiviral vector” and “lentiviral expression vector” may be used to refer to lentiviral transfer plasmids and/or infectious lentiviral particles. Where reference is made herein to elements such as cloning sites, promoters, regulatory elements, heterologous nucleic acids, etc., it is to be understood that the sequences of these elements are present in RNA form in the lentiviral particles of the invention and are present in DNA form in the DNA plasmids of the invention.

According to certain specific embodiments, most or all of the viral vector backbone sequences are derived from a lentivirus, e.g., HIV-1. However, it is to be understood that many different sources of lentiviral sequences can be used, and numerous substitutions and alterations in certain of the lentiviral sequences may be accommodated without impairing the ability of a transfer vector to perform the functions described herein. Moreover, a variety of lentiviral vectors are known in the art, see Naldini et al., (1996a, 1996b, and 1998); Zufferey et al., (1997); Dull et al., 1998, U.S. Pat. Nos. 6,013,516; and 5,994,136, many of which may be adapted to produce a viral vector or transfer plasmid of the present invention.

In preparing lentiviral vector, any host cells for producing lentiviral vectors may be employed, including, for example, mammalian cells (e.g. HEK 293T cells). Host cells can also be packaging cells in which the lentiviral gag/pol and rev genes are stably maintained in the host cell or producer cells in which the lentiviral vector genome is stably maintained and packaged. Lentiviral vectors are purified and formulated using standard techniques known in the art.

In certain embodiments, the present invention includes a cell comprising a gene expression cassette, gene transfer cassette, or recombinant lentiviral vector disclosed herein. In related embodiments, the cell is transduced with a recombinant lentiviral vector comprising an expression cassette disclosed herein or has an expression cassette disclosed herein integrated into the cell's genome. In certain embodiments, the cell is a cell used to produce a recombinant retroviral vector, e.g., a packaging cell.

In some embodiments, the lentiviral vector is pseudotyped. For example, a plasmid comprising a heterologous env gene can be used for pseudotyping. Suitable env genes include, without limitation, VSV-G.

In some embodiments, the backbone of the transfer plasmid comprises an RNA-OUT sequence. RNA-OUT is a selectable marker system that facilitates selection of cells harboring the transfer plasmid within the use of antibiotics, as described, e.g., in U.S. Pat. Nos. 9,109,012 and 9,737,620, which are incorporated by reference herein. In some embodiments, the RNA-OUT sequence is:

(SEQ ID NO: 22)
GTAGAATTGGTAAAGAGAGTCGTGTAAAATATCGA
GTTCGCACATCTTGTTGTCTGATTATTGATTTTTG
GCGAAACCATTTGATCATATGACAAGATGTGTATC
TACCTTAACTTAATGATTTTGATAAAAATCATTAG
G

Advantageously, the RNA-OUT sequence contributes to stable propagation of the transfer plasmid in a packing cell line.

In some embodiments, the disclosure provides a transfer the expression cassette comprises a polynucleotide that shares at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity with SEQ ID NO: 1. In some embodiments, the expression cassette comprises a polynucleotide that shares at least 80%, 85%, 90%, 95%, 99%, or 100% identity with SEQ ID NO: 1. In some embodiments, the expression cassette has the sequence SEQ ID NO: 1.

AAV is a 4.7 kb, single stranded DNA virus. Recombinant vectors based on AAV are associated with excellent clinical safety, since wild-type AAV is nonpathogenic and has no etiologic association with any known diseases. In addition, AAV offers the capability for highly efficient gene delivery and sustained transgene expression in numerous tissues. By an “AAV vector” is meant a vector derived from an adeno-associated virus serotype, including without limitation, AAV1, AAV2, AAV3, AAV4, AAV5, AAV6, AAV7, AAV8, AAV9, AAV10, AAVrh.10, AAVrh.74, etc. AAV vectors can have one or more of the AAV wild-type genes deleted in whole or part, e.g., the rep and/or cap genes, but retain functional flanking inverted terminal repeat (ITR) sequences. Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g. by the insertion, deletion or substitution of nucleotides, as long as the sequences provide for functional rescue, replication and packaging. AAV vectors may comprise other modifications, including but not limited to one or more modified capsid protein (e.g., VP1, VP2 and/or VP3). For example, a capsid protein may be modified to alter tropism and/or reduce immunogenicity. AAV expression vectors are constructed using known techniques to at least provide as operatively linked components in the direction of transcription, control elements including a transcriptional initiation region, the DNA of interest (i.e. the TCIRG1 gene) and a transcriptional termination region.

### Pharmaceutical Compositions and Methods of Use

The present disclosure also provides pharmaceutical compositions comprising an expression cassette or vector (e.g., gene therapy vector) disclosed herein and one or more pharmaceutically acceptable carriers, diluents or excipients. In some embodiments, the pharmaceutical composition comprises a lentiviral particle comprising an expression cassette disclosed herein, e.g., wherein the expression cassette comprises a codon-transgene encoding TCIRG1, e.g., SEQ ID NOs: 3. Provided are pharmaceutical compositions, e.g., for use in preventing or treating a disorder characterized by deficient osteoclast formation (e.g., Infantile Malignant Osteopetrosis) which comprises a therapeutically effective amount of a lentiviral particle that comprises a nucleic acid sequence of a polynucleotide that encodes one or more isoforms of TCIRG1.

In particular embodiments, the lentiviral particles disclosed herein are used to transduce autologous CD34+ hematopoietic stem cells (HSCs) derived from a subject, thus complementing the genetic defect. Transduction may occur in vivo or ex vivo. The CD34+ enriched cell population is cultured, in some embodiments, in CellGenix Stem Cell Growth Media (SCGM) with recombinant human cytokines and incubated in 5% CO₂ and 5% O₂ at 37° C. The CD34+ enriched cell population is, in some embodiments, incubated with the same additives as used for the pre-stimulation, optionally with the addition of transduction enhancers, and lentiviral particles comprising the expression cassette EFS-TCIRG1-WPRE (at, for example, MOI 50). Following transduction, the cell suspension is, in some embodiments, washed a portion of cells and supernatant are removed for release testing and the drug product is frozen in preparation for infusion. In some embodiments, HSC are mobilized by treating the patient with G-CSF, plerifaxor, or a combination of G-CSF and plerifaxor. The HSCs are then collected from peripheral blood of the patient by apheresis. CD34+ cells are enriched, e.g., using magnetic capture (e.g., on the Miltenyi Biotec CliniMACs system), and the CD34+ enriched cells are transduced ex vivo with the lentiviral particles. In some embodiments, the transduction process incorporates the use of transductions enhancers, such as, without limitation, polyaxamers and Prostaglandin E2 (PGE2).

In some embodiments, the transduced HSCs are then transplanted into a subject, e.g., a human subject, by infusion with at least 2.0×10⁶ CD34+ cells/kg. In some embodiments, they repopulate the HSC niche with TCIRG1-expressing cells. In some embodiments, they repopulate the osteoclast niche with TCIRG1-expressing cells.

Provided also are pharmaceutical compositions, e.g., for use in preventing or treating a disorder characterized by deficient osteoclast formation (e.g., Infantile Malignant Osteopetrosis) which comprises a therapeutically effective amount of a modified cell that comprises a nucleic acid sequence of a polynucleotide that encodes one or more isoforms of TCIRG1. In some embodiments, the modified cell expresses TCIRG1 or a functional variant thereof at a level similar to the level of expression of TCIRG1 observed in an osteoclast having a functional TCIRG1 gene. In some embodiments, the modified cell expresses TCIRG1 or a functional variant thereof at a level similar to the level of expression of TCIRG1 observed in an osteoclast derived from a subject not having or suspected of having IMO. In some embodiments, the modified cell is a hematopoietic stem cell (HSC). In some embodiments, the modified cell is a CD34+ progenitor cell. In some embodiments, the modified cell is derived a HSC isolated from a subject having or suspected of having IMO by apheresis. In some embodiments, the modified cell is autologous to the subject. In some embodiments, the modified cell is derived a HSC isolated from a subject having or suspected of having IMO by apheresis after mobilization of HSCs by administration of G-CSF, plerifaxor, or a combination of G-CSF and plerifaxor. In some embodiments, the modified cell is derived from a population of cells enriched for CD34+ cells by magnetic capture. In some embodiments, the modified cell was transduced using a vector disclosed herein, e.g., a lentiviral vector produced using a transfer plasmid disclosed herein.

The pharmaceutical compositions that contain the expression cassette or lentiviral particle or modified cell may be in any form that is suitable for the selected mode of administration, for example, for intraventricular, intramyocardial, intracoronary, intravenous, intra-arterial, intra-renal, intraurethral, epidural or intramuscular administration. The gene modified cell comprising a polynucleotide encoding one or more TCIRG1 isoforms can be administered as sole active agent, or in combination with other active agents, in a unit administration form, as a mixture with conventional pharmaceutical supports, to animals and human beings. In some embodiments, the pharmaceutical composition comprises cells transduced ex vivo with any of the gene therapy vectors of the disclosure.

In various embodiments, the pharmaceutical compositions contain vehicles (e.g., carriers, diluents and excipients) that are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. Illustrative pharmaceutical forms suitable for injectable use include, e.g., sterile aqueous solutions or dispersions; formulations including sesame oil, peanut oil or aqueous propylene glycol; and sterile powders for the extemporaneous preparation of sterile injectable solutions or dispersions.

In another aspect, the disclosure provides methods of preventing, mitigating, ameliorating, reducing, inhibiting, eliminating and/or reversing one or more symptoms of Infantile Malignant Osteopetrosis (IMO) or another disorder in a subject in need thereof, comprising administering to the subject a gene therapy vector of the disclosure. The term “Infantile Malignant Osteopetrosis” or “malignant infantile osteopetrosis” or “infantile autosomal recessive osteopetrosis” or “infantile osteopetrosis” or “IMO” refers to a rare osteosclerosis type of skeletal dysplasia that typically presents in infancy and is characterized by a unique radiographic appearance of generalized hyperostosis—excessive growth of bone. The generalized increase in bone density has a special predilection to involve the medullary portion with relative sparing of the cortices. Obliteration of bone marrow spaces and subsequent depression of the cellular function can result in serious hematologic complications. Optic atrophy and cranial nerve damage secondary to bony expansion can result in marked morbidity. The prognosis is extremely poor in untreated cases Plain radiography provides the key information to the diagnosis. Clinical and radiologic correlations are also fundamental to the diagnostic process, with additional gene testing being confirmatory.

In an embodiment, the modified cell, e.g. an autologous cell transduced with a lentiviral particle of the disclosure, is administered via a route selected from the group consisting of parenteral, intravenous, intra-arterial, intracardiac, intracoronary, intramyocardial, intrarenal, intraurethral, epidural, and intramuscular. In some embodiments, the modified cells is administered by infusion, e.g. intravenous infusion. In an embodiment, modified cells are administered multiple times. In an embodiment, modified cells are administered by infusion.

In an embodiment, the disclosure provides a method of treating a disease or disorder, optionally IMO, in a subject in need thereof, comprising contacting cells with a gene therapy vector according to the present disclosure and administering the cells to the subject. In an embodiment, the cells are stem cells, optionally pluripotent stem cells. In an embodiment, the stem cells are capable of differentiation into bone cells. In an embodiment, the stem cells are capable of differentiation into osteoclasts. In an embodiment, the stem cells are autologous. In an embodiment, the stem cells are CD34+ stem cells.

In an embodiment, the subject is exhibiting symptoms of IMO or another disorder. In an embodiment, the subject has been identified as having reduced or non-detectable TCIRG1 expression. In an embodiment, the subject has been identified as having a mutated TCIRG1 gene.

Subjects/patients amenable to treatment using the methods described herein include individuals at risk of a disease or disorder characterized by insufficient osteoclasts (e.g., IMO as well as other known disorders of osteoclast formation. In some embodiments, the subject is not showing symptoms. In some embodiments, subjects is presently showing symptoms. Such subject may have been identified as having a mutated TCIRG1 gene or as having reduced or non-detectable levels of TCIRG1 expression. The symptoms may be actively manifesting, or may be suppressed or controlled (e.g., by medication) or in remission. The subject may or may not have been diagnosed with the disorder, e.g., by a qualified physician.

### Definitions

The terms “T cell immune regulator 1, ATPase H+ transporting V0 subunit a3” and “TCIRG1” interchangeably refer to nucleic acids and polypeptide polymorphic variants, alleles, mutants, and interspecies homologs that: (1) have an amino acid sequence that has greater than about 90% amino acid sequence identity, for example, 91%, 92%, 93%, 94%, 95%, 96%, 97%, 98% or 99% or greater amino acid sequence identity, preferably over a region of at least about 25, 50, 100, 200, 300, 400, or more amino acids, or over the full-length, to an amino acid sequence encoded by a TCIRG1 nucleic acid (see, e.g., GenBank Accession Nos. NM_006019.4 (variant 1). NM_006053.3 (variant 2), NM_001351059.1 (variant 3)) or to an amino acid sequence of a TCIRG1 polypeptide (see e.g., GenBank Accession Nos. NP_006044.1 (isoform A), NP_006044.1 (isoform B), NP_001337988.1 (isoform C)); (2) bind to antibodies, e.g., polyclonal antibodies, raised against an immunogen comprising an amino acid sequence of a TCIRG1 polypeptide (e.g., TCIRG1 polypeptides described herein); or an amino acid sequence encoded by a TCIRG1 nucleic acid (e.g., TCIRG1 polynucleotides described herein), and conservatively modified variants thereof; (3) specifically hybridize under stringent hybridization conditions to an anti-sense strand corresponding to a nucleic acid sequence encoding a TCIRG1 protein, and conservatively modified variants thereof; (4) have a nucleic acid sequence that has greater than about 90%, preferably greater than about 91%, 92%, 93%, 94%, 95%, 96%, 97%, 98%, 99%, or higher nucleotide sequence identity, preferably over a region of at least about 25, 50, 100, 200, 500, 1000, 2000 or more nucleotides, or over the full-length, to a TCIRG1 nucleic acid (e.g., TCIRG1 polynucleotides, as described herein, and TCIRG1 polynucleotides that encode TCIRG1 polypeptides, as described herein).

The TCIRG1 gene encodes several protein isoforms, with 2 main isoforms. The full-length isoform a (OC116) encodes the A3 subunit of vacuolar H(+)-ATPase, which is involved in regulation of the pH of intracellular compartments and organelles of eukaryotic cells, including the pH of intracellular compartments and organelles of osteoclasts. The shorter isoform b (TIRC7) encodes a T-cell-specific membrane protein that plays an essential role in T-lymphocyte activation and immune response.

The terms “identical” or percent “identity,” in the context of two or more nucleic acids or polypeptide sequences, refer to two or more sequences or subsequences that are the same or have a specified percentage of amino acid residues or nucleotides that are the same (i.e., share at least about 80% identity, for example, at least about 85%, 90%, 91%, 92%, 93%, 94%, 95%, 96%, 97%, 98%, 99% identity over a specified region to a reference sequence, e.g., TCIRG1 polynucleotide or polypeptide sequence as described herein, when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Preferably, the identity exists over a region that is at least about 25 amino acids or nucleotides in length, for example, over a region that is 50, 100, 200, 300, 400 amino acids or nucleotides in length, or over the full-length of a reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins to TCIRG1 nucleic acids and proteins, the BLAST and BLAST 2.0 algorithms and the default parameters are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, W1), or by manual alignment and visual inspection (see, e.g., Ausubel et al., eds., Current Protocols in Molecular Biology (1995 supplement)). Examples of algorithms that are suitable for determining percent sequence identity and sequence similarity are the BLAST and BLAST 2.0 algorithms, which are described in Altschul et al., Nucleic Acids Res. 25:3389-3402 (1977) and Altschul et al., J. Mol. Biol. 215:403-410 (1990), respectively. Software for performing BLAST analyses is publicly available through the National Center for Biotechnology Information (on the worldwide web at ncbi.nlm.nih.gov).

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions. Yet another indication that two nucleic acid sequences are substantially identical is that the same primers can be used to amplify the sequence.

As used herein, “administering” refers to local and systemic administration, e.g., including enteral, parenteral, pulmonary, and topical/transdermal administration. Routes of administration for compounds (e.g., polynucleotide encoding one or more TCIRG1 isoforms) that find use in the methods described herein include, e.g., oral (per os (P.O.)) administration, nasal or inhalation administration, administration as a suppository, topical contact, transdermal delivery (e.g., via a transdermal patch), intrathecal (IT) administration, intravenous (“iv”) administration, intraperitoneal (“ip”) administration, intramuscular (“im”) administration, intralesional administration, or subcutaneous (“sc”) administration, or the implantation of a slow-release device e.g., a mini-osmotic pump, a depot formulation, etc., to a subject. Administration can be by any route including parenteral and transmucosal (e.g., oral, nasal, vaginal, rectal, or transdermal). Parenteral administration includes, e.g., intravenous, intramuscular, intraarterial, intrarenal, intraurethral, intracardiac, intracoronary, intramyocardial, intradermal, epidural, subcutaneous, intraperitoneal, intraventricular, ionophoretic and intracranial. Other modes of delivery include, but are not limited to, the use of liposomal formulations, intravenous infusion, transdermal patches, etc.

The terms “systemic administration” and “systemically administered” refer to a method of administering a compound or composition to a mammal so that the compound or composition is delivered to sites in the body, including the targeted site of pharmaceutical action, via the circulatory system. Systemic administration includes, but is not limited to, oral, intranasal, rectal and parenteral (e.g., other than through the alimentary tract, such as intramuscular, intravenous, intra-arterial, transdermal and subcutaneous) administration.

The term “co-administering” or “concurrent administration”, when used, for example with respect to the compounds (e.g., TCIRG1 polynucleotides) and/or analogs thereof and another active agent, refers to administration of the compound and/or analogs and the active agent such that both can simultaneously achieve a physiological effect. The two agents, however, need not be administered together. In certain embodiments, administration of one agent can precede administration of the other. Simultaneous physiological effect need not necessarily require presence of both agents in the circulation at the same time. However, in certain embodiments, co-administering typically results in both agents being simultaneously present in the body (e.g., in the plasma) at a significant fraction (e.g., 20% or greater, e.g., 30% or 40% or greater, e.g., 50% or 60% or greater, e.g., 70% or 80% or 90% or greater) of their maximum serum concentration for any given dose.

The term “effective amount” or “pharmaceutically effective amount” refer to the amount and/or dosage, and/or dosage regime of one or more compositions (e.g., gene therapy vectors, modified cells) necessary to bring about the desired result e.g., increased expression of one or more TCIRG1 isoforms in an amount sufficient to reduce the ultimate severity of a disease characterized by impaired or deficient autophagy (e.g., IMO).

The phrase “cause to be administered” refers to the actions taken by a medical professional (e.g., a physician), or a person controlling medical care of a subject, that control and/or permit the administration of the agent(s)/compound(s) at issue to the subject. Causing to be administered can involve diagnosis and/or determination of an appropriate therapeutic or prophylactic regimen, and/or prescribing particular agent(s)/compounds for a subject. Such prescribing can include, for example, drafting a prescription form, annotating a medical record, and the like.

As used herein, the terms “treating” and “treatment” refer to delaying the onset of, retarding or reversing the progress of, reducing the severity of, or alleviating or preventing either the disease or condition to which the term applies, or one or more symptoms of such disease or condition. The terms “treating” and “treatment” also include preventing, mitigating, ameliorating, reducing, inhibiting, eliminating and/or reversing one or more symptoms of the disease or condition.

The term “mitigating” refers to reduction or elimination of one or more symptoms of that pathology or disease, and/or a reduction in the rate or delay of onset or severity of one or more symptoms of that pathology or disease, and/or the prevention of that pathology or disease. In certain embodiments, the reduction or elimination of one or more symptoms of pathology or disease can include, e.g., measurable and sustained increase in the expression levels of one or more isoforms of TCIRG1.

As used herein, the phrase “consisting essentially of refers to the genera or species of active pharmaceutical agents recited in a method or composition, and further can include other agents that, on their own do not have substantial activity for the recited indication or purpose.

The terms “subject,” “individual,” and “patient” interchangeably refer to a mammal, preferably a human or a non-human primate, but also domesticated mammals (e.g., canine or feline), laboratory mammals (e.g., mouse, rat, rabbit, hamster, guinea pig) and agricultural mammals (e.g., equine, bovine, porcine, ovine). In various embodiments, the subject can be a human (e.g., adult male, adult female, adolescent male, adolescent female, male child, female child).

The terms “gene transfer” or “gene delivery” refer to methods or systems for reliably inserting foreign DNA into host cells. Such methods can result in transient expression of non-integrated transferred DNA, extrachromosomal replication and expression of transferred replicons (e.g. episomes), or integration of transferred genetic material into the genomic DNA of host cells.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication or reverse transcription in a cell, or may include sequences sufficient to allow integration into host cell DNA. “vectors” include gene therapy vectors. As used herein, the term “gene therapy vector” refers to a vector capable of use in performing gene therapy, e.g., delivering a polynucleotide sequence encoding a therapeutic polypeptide to a subject. Gene therapy vectors may comprise a nucleic acid molecule (“transgene”) encoding a therapeutically active polypeptide, e.g., a TCIRG1 or other gene useful for replacement gene therapy when introduced into a subject. Useful vectors include, but are not limited to, viral vectors.

As used herein, the term “expression cassette” refers to a DNA segment that is capable in an appropriate setting of driving the expression of a polynucleotide (e.g., a transgene) encoding a therapeutically active polypeptide (e.g., TCIRG1) that is incorporated in said expression cassette. When introduced into a host cell, an expression cassette inter alia is capable of directing the cell's machinery to transcribe the transgene into RNA, which is then usually further processed and finally translated into the therapeutically active polypeptide. The expression cassette can be comprised in a gene therapy vector. Generally, the term expression cassette excludes polynucleotide sequences 5′ to the 5′ LTR and 3′ to the 3′ LTR.

All patents, patent publications, and other publications referenced and identified in the present specification are individually and expressly incorporated herein by reference in their entirety for all purposes.

### EXAMPLES

### Example 1: Stable Propagation of Transfer Plasmids

The stability of different plasmids comprising the minimal TCIRG1 expression cassette, EFS-TCIRG1-WPRE (SEQ ID NO: 1) was examined. The plasmid construct pRRL.PPT.EFS.tcirg1h.wpre (FIG. 3C) with ampicillin resistance (AmpR) showed unexpectedly poor growth and instability during culture in E. coli cells used to propagate the plasmid prior to transfection into a packaging cell line, as shown by low yield of plasmid and a general smear of degraded DNA indicative of instability (FIG. 3A). Various other plasmid backbones also showed instability (data not shown). However, when the minimal expression cassette EFS-TCIRG1-WPRE (SEQ ID NO: 1) was cloned from the pRRL vector into a pCCL vector with an RNA-OUT sequence (FIG. 3C), the resulting plasmid construct, pCCL.PPT.EFS.tcirg1h.wpre (SEQ ID NO: 27), exhibited unexpectedly good growth and stability when propagated in E. coli. This was shown by high yield of plasmid and the restriction digest pattern observed (FIG. 3B). The full vector sequence is provided as SEQ ID NO: 27, with the positions of each vector element provided in Table 2.

TABLE 2
pCCL.PPT.EFS.tcirg1h.wpre Vector Elements

| | | Min. - Max. | |
|---|---|---|---|
| | | Position in | Length |
| | | Reference Sequence | (base |
| Name | Type | (nucleotide) | pairs) |
| EFS | Promoter | 1-243 | 243 |
| TCIRG1 | CDS | 257-2,749 | 2,493 |
| WPRE | Regulatory | 2,782-3,384 | 603 |
| 3′LTR | LTR | 3,471-3,704 | 234 |
| SV40 poly(A) | polyA signal | 3,776-3,907 | 132 |
| SV40 ori | Origin of replication | 3,917-4,076 | 160 |
| pUC origin | Origin of replication | 4,115-5,129 | 1,015 |
| RNA-OUT | Repressor | 5,146-5,284 | 139 |
| CMV IE | Promoter | 5,334-5,910 | 577 |
| 5-LTR | LTR | 5,933-6,120 | 188 |
| psi | Packaging | 6,222-6,266 | 45 |
| gag | CDS | 6,267-6,628 | 362 |
| RRE | Regulatory | 6,629-7,486 | 858 |
| cPPT/CTS | Poly purine tract | 7,505-7,622 | 118 |

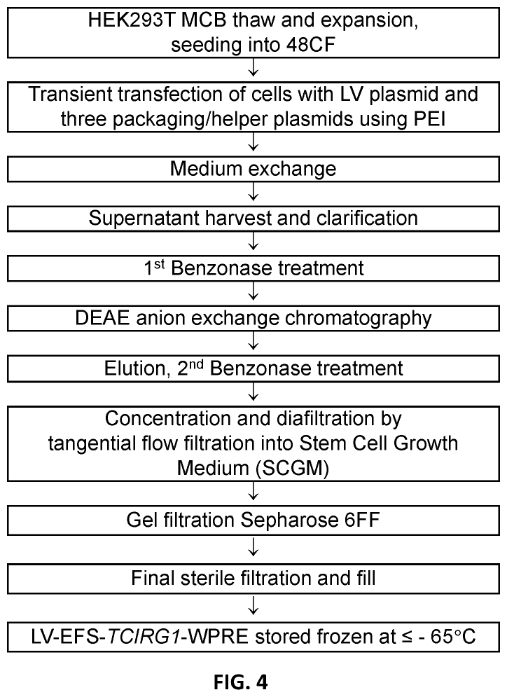

Lentiviral vectors are produced by transient transfection of the pCCL/RNA-OUT vector into 293T cells along with packaging plasmid (pCMV ΔR8.91), and envelope plasmid (VSV-G pMDG) and produced according to the protocol depicted in FIG. 4.

### Example 2: Restoration of Resorptive Function of Osteoclasts from IMO Patients with pCCL.PPTEFS.tcirg1h.wpre

This example demonstrates use of the pCCL.PPT.EFS.tcirg1h.wpre for lentiviral-mediated TCIRG1 gene transfer in patient-derived HSCs. HSCs are obtained, expanded and transduced with lentiviral particles carrying the pCCL.PPT.EFS.tcirg1h.wpre described in Example 1 to obtain gene-modified HSCs. Following infusion, the gene-modified HSCs will differentiate into osteoclasts. Methods used are essentially as described in Moscatelli et al. Hum. Gene Therap. 29:938-949 (2017).

Samples of peripheral blood from IMO patients or umbilical cord blood (CB) from normal deliveries are obtained. Mononuclear cells from these sources are isolated using density gradient centrifugation with Ficoll and CD34+ cells are separated from the mononuclear cell fraction using magnet-activated cell sorting (MACS) columns (Miltenyi Biotec, Bergisch Gladbach, Germany). For expansion, cells are cultured in SFEM StemSpan medium (StemCell Technologies, Vancouver, BC) with the human recombinant cytokines M-CSF (50 ng/ml), GM-CSF (30 ng/ml), SCF (200 ng/ml), IL-6 (10 ng/ml) and Flt3L (50 ng/ml) (R&D Systems, Minneapolis Minn.). CD34+ cells are plated at a density of 5×10⁴ cells in 1 ml medium using 24-well bacteriological plates and incubated for a week at 37° C. before collection and replating at a density of 1×10⁵/well. From day 7 the medium is exchanged every 2-3 days by demi-depletion. For transplantation, CD34+ cells are cultured for 30 hours in SFEM StemSpan medium (StemCell Technologies, Vancouver, BC) with the human recombinant cytokines: SCF (100 ng/ml), Flt3L (100 ng/ml) and TPO (100 ng/ml) (R&D Systems, Minneapolis Minn.).

Transductions are carried out in 24-well plates coated with RetroNectin (Takara Bio, Otsu, Japan). For the in vitro experiments CD34+ cells were transduced with a first hit at a multiplicity of infection (MOI) of 30 for 6 hours on day 3 and a second hit at MOI 30 for 6 hours on day 7 followed by a week of culture with a myeloid cytokine cocktail and subsequent differentiation to osteoclasts. For the in vivo experiments, a shorter transduction protocol is used to allow efficient transduction while maintaining the stem/progenitor nature of the CD34+ population. Mononuclear cells are isolated and transduced with the first hit (MOI 30 or 100) overnight, followed by transduction on the following day with a second hit (MOI 30 or 100) for six hours, after which the cells are ready for transplanted into subjects (mice or human patients).

Osteoclastogenesis can be assessed by differentiation for about ten days in the presence of 50 ng/ml M-CSF and 50 ng/ml RANKL, followed by fixation of the cells with 4% formaldehyde for further analyzes or lysis of cells for western blot analysis. Resorption is assessed by assaying for release c-terminal type I collagen fragments (CTX-I) and for release of Ca2+ into the media, and by visualization of the formation of resorption pits using hematoxylin staining of fixed cells.

For animal studies, transduced osteoclasts are transplanted into NSG mice. NSG mice, 8 to 15 week old, are sublethally irradiated with 300 cGy and transplanted six hours later with 1×10⁵ untransduced CB CD34+ cells or IMO CD34+ cells transduced with lentiviral particles derived from pCCL.PPT.EFS.tcirg1h.wpre, by tail vein injection. The mice are administered ciprofloxacin via their drinking water for two weeks to avoid post-transplantation infections. Peripheral blood was harvested at different time points and bone marrow cells are harvested by crushing the femora with a mortar after termination of the mice.

Vector copy number analysis is performed on whole bone marrow genomic DNA from samples harvested from mice 9-19 weeks after transplantation. Peripheral blood and bone marrow of transplanted NSG mice are analyzed for human reconstitution by determining the percentage of cells positive for huCD45-APC. For lineage analysis, the cells were stained with antibodies directed against CD33-PeCy7, CD15-PeCy7, CD19-BV605 and CD3-PE.

The methods described above are used to confirm restoration of resorptive function of osteoclasts from IMO patients after lentiviral-mediated TCIRG1 gene transfer and long-term engraftment of transduced CD34+ cells.

### Example 3: In Vitro Transduction of Human CD34⁺ Enriched Cells Using a Clinically Established Transduction Protocol

This example demonstrates suitability of a pre-GMP batch of EFS-TCIRG1-WPRE (SEQ ID NO: 25 or 27) made using the plasmid described in Example 1 by: (1) Phenotypic characterization of transduced CD34⁺ cells by means of % viable CD34⁺ cells and multilineage differentiation capacity, and (2) Transduction efficiency by means of vector copy number (VCN determination both in liquid culture and colonies.

Preserved Phenotype and Multilineage Capacity of mPB CD34+ Cells after Transduction with EFS-TCIRG1-WPRE

The performance of a pre-GMP EFS-TCIRG1-WPRE batch was compared with the LVs produced for the treatment of other disorders (four batches). Mobilized PB CD34⁺ cells were used as target cells similarly as in the envisioned IMO clinical trial. Various MOIs were tested. High cell viability 20 hours after transduction (>95%) was obtained across all vectors and MOIs tested, indicating no short-term toxicity. The percentage of CD34⁺ cells was very high for all conditions tested (>97%) shortly after transduction, and after 2 days in liquid culture was progressively lost over time at comparable levels in all conditions, as expected for this type of culture.

Multilineage capacity of transduced CD34⁺ cells was evaluated by means of quantification of differentiated CFUs in semisolid methylcellulose medium cultures. Total CFUs as well as BFU-E, CFU-GM, and CFU-GEMM, accounting for the erythroid and myeloid lineages, were evaluated. The presence of the EFS-TCIRG1-WPRE did not affect CFU growth as no significant differences in comparison with a Mock control were observed in their total numbers among experimental conditions. No differences in comparison with the Mock were observed in any colony type thus confirming the maintenance of multilineage capacity in vitro after transduction with the EFS-TCIRG1-WPRE even at high MOI values.

### EFS-TCIRG1-WPRE Shows High Transduction Efficiency of mPB CD34+ Cells

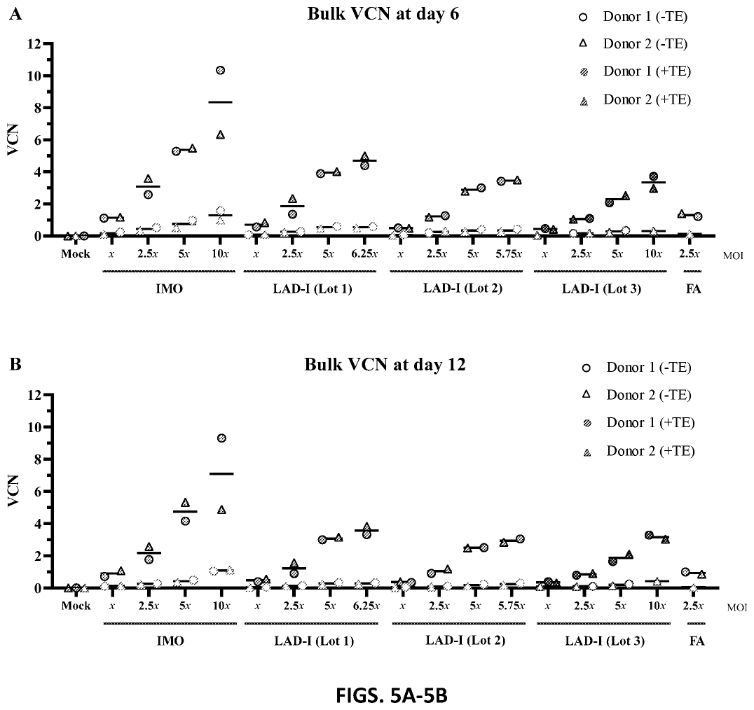

To determine the vector dose of EFS-TCIRG1-WPRE required to provide suitable transduction efficiency for therapeutic use, lentiviral vectors were tested using the established clinical transduction protocol. Transduced CD34⁺ cells were maintained in liquid culture for up to 12 days to allow for cellular clearance of episomal LV genome copies prior to VCN assessment. High VCN/cell values were obtained with the IMO vector that were dose-dependent. The effects of increasing dose were consistent across all vectors tested, and transduction with the same MOI resulted in higher VCN/cell values for the IMO vector than for LAD-I and FA vectors (FIGS. 5A-5B), indicating high transduction efficiency of EFS-TCIRG1-WPRE.

VCN/cell was also evaluated in isolated CFUs depending on their phenotype: BFU-E, CFU-GM, or CFU-GEMM, to confirm transduction in different progenitors. EFS-TCIRG1-WPRE showed higher colony VCN values in cells from both donors, Similar to results in liquid culture, transduction with IMO vector resulted in high transduction efficiency and VCN/cell of colonies cultured in methylcellulose medium.

VCN pattern in the different CFU types (BFU-E, CFU-GM, and CFU-GEMM) was found to be similar to the other vectors and with the highest values usually found in the erythroid colonies as previously described in Charrier et al. Gene Therapy 18:479-487(2011).

The IMO vector EFS-TCIRG1-WPRE transduces human CD34⁺ cells at levels comparable to clinical lots of lentivirus. The phenotype of CD34⁺ cells and multilineage capacity were preserved while high transduction efficiency was achieved. IMO vector performed successfully at lower MOI than control vectors, demonstrating its suitability for use in the gene therapy of patients with IMO.

These studies demonstrated that a VCN and transduction efficiency was achieved that parallels corrective levels of in vivo gene-modified hematopoietic cells, enabling use of this gene therapy in the treatment of infantile malignant osteopetrosis due to mutations in TCIRG1.

What is claimed is:

1. A transfer plasmid, comprising an expression cassette comprising a coding polynucleotide encoding an isoform of T cell immune regulator 1 (TCIRG1) or a functional variant thereof, and a promoter, wherein the polynucleotide is operatively linked to the promoter, and wherein the transfer plasmid comprises an RNA-OUT repressor and a CMV IE promoter.

2. The transfer plasmid of claim 1, wherein the RNA-OUT repressor shares at least 95% identity or at least 99% identity to SEQ ID NO: 32.

3. The transfer plasmid of claim 1 or claim 2, wherein the CMV IE promoter shares at least 95% identity or at least 99% identity to SEQ ID NO: 33.

4. The transfer plasmid of any one of claims 1 to 3, wherein the transfer plasmid comprises a pCCL backbone

5. The transfer plasmid of claim 4, wherein the pCCL backbone comprises the RNA-OUT repressor.

6. The transfer plasmid of claim 5, wherein the transfer plasmid shares at least 95% or 100% identity to SEQ ID NO: 39.

7. The transfer plasmid of any one of claims 1 to 7, wherein the promoter is an EFS promoter.

8. The transfer plasmid of claim 7, wherein the EFS promoter shares at least 95% identity with SEQ ID NO: 2.

9. The transfer plasmid of claim 8, wherein the EFS promoter is SEQ ID NO: 2.

10. The transfer plasmid of any one of claims 1 to 9, wherein the coding polynucleotide shares at least 95% identity with SEQ ID NO: 3.

11. The transfer plasmid of claim 10, wherein the coding polynucleotide shares at least 99% identity with SEQ ID NO: 3.

12. The transfer plasmid of claim 11, wherein the coding polynucleotide is SEQ ID NO: 3.

13. The transfer plasmid of any one of claims 1 to 12, wherein the expression cassette comprises a Woodchuck Hepatitis Virus (WHP) Posttranscriptional Regulatory Element (WPRE).

14. The transfer plasmid of claim 13, wherein the WPRE is SEQ ID NO: 4.

15. The transfer plasmid of any one of claims 1 to 14, wherein the expression cassette shares at least 95% identity with SEQ ID NO: 1.

16. The transfer plasmid of any one of claims 1 to 15, wherein the expression cassette is flanked by a 5′ long terminal repeat (LTR) and a 3′ LTR.

17. The transfer plasmid of claim 16, wherein the 5′ LTR is SEQ ID NO: 34 and/or the 3′ LTR is SEQ ID NO: 28.

18. The transfer plasmid of any one of claims 1 to 17, wherein expression cassette shares at least 95% identity to SEQ ID NO: 1.

19. The transfer plasmid of any one of claims 1 to 17, wherein expression cassette is SEQ ID NO: 1.

20. A lentiviral particle produced by transfecting a host cell with the transfer plasmid of any one of claims 1 to 20.

21. An expression cassette comprising a coding polynucleotide encoding an isoform of T cell immune regulator 1 (TCIRG1) or a functional variant thereof, and an EFS promoter, wherein the polynucleotide is operatively linked to the EFS promoter.

22. The expression cassette of claim 1, wherein the coding polynucleotide shares at least 95% identity with SEQ ID NO: 3.

23. The expression cassette of claim 2, wherein the coding polynucleotide shares at least 99% identity with SEQ ID NO: 3.

24. The expression cassette of claim 3, wherein the coding polynucleotide is SEQ ID NO: 3.

25. The expression cassette of any one of claims 1 to 4, wherein the EFS promoter shares at least 95% identity with SEQ ID NO: 2.

26. The expression cassette of claim 25, wherein the EFS promoter is SEQ ID NO: 2.

27. The expression cassette of any one of claims 21 to 26, comprising a Woodchuck Hepatitis Virus (WHP) Posttranscriptional Regulatory Element (WPRE).

28. The expression cassette of claim 27, wherein the WPRE is SEQ ID NO: 4.

29. The expression cassette of any one of claims 21 to 28, wherein the expression cassette shares at least 95% identity with SEQ ID NO: 1.

30. The expression cassette of claim 29, wherein the expression cassette is SEQ ID NO: 1.

31. A recombinant lentiviral genome, comprising in 5′ to 3′ order:
(a) a lentiviral 5′ long terminal repeat (LTR);
(b) the expression cassette of any one of claims 21 to 30; and
(c) a lentiviral 3′LTR,
wherein the recombinant lentiviral genome is replication incompetent.

32. A transfer plasmid, comprising the recombinant lentiviral genome of claim 31.

33. A lentiviral particle, comprising the recombinant lentiviral genome of claim 31.

34. A pharmaceutical composition, comprising the lentiviral particle of claim 33.

35. A modified cell, comprising the expression cassette of any one of claims 21 to 30.

36. A modified cell, comprising the recombinant lentiviral genome of claim 31.

37. The modified cell of claim 36, wherein the modified cell lacks an endogenous functional TCIRG1 gene.

38. The modified cell of claim 36 or 37, wherein the modified cell is derived from a subject having or suspected of having infantile malignant osteopetrosis (IMO).

39. The modified cell of any of one claims 36 to 38, wherein the modified cell expresses TCIRG1 or a functional variant thereof at a level similar to the level of expression of TCIRG1 observed in an osteoclast having a functional TCIRG1 gene.

40. The modified cell of any of one claims 36 to 39, wherein the modified cell expresses TCIRG1 or a functional variant thereof at a level similar to the level of expression of TCIRG1 observed in an osteoclast derived from a subject not having or suspected of having IMO.

41. The modified cell of any of one claims 36 to 40, wherein the modified cell is a hematopoietic stem cell (HSC).

42. The modified cell of any of one claims 36 to 41, wherein the modified cell is a CD34+ progenitor cell.

43. The modified cell of any of on claim 41 or 42, wherein the modified cell is derived from a HSC isolated from a subject having or suspected of having IMO by apheresis, optionally after mobilization of HSCs by administration of G-CSF, plerifaxor, or a combination of G-CSF and plerifaxor.

44. The modified cell of any one of claims 35 to 43, wherein the modified cell is derived from a population of cells enriched for CD34+ cells by magnetic capture.

45. A pharmaceutical composition comprising the modified cell of any one of claims 35 to 44.

46. An in vitro method of modifying one or more cells of a subject having or suspected of having IMO, comprising:
(a) providing peripheral blood mononuclear cells (PBMCs) mobilized from the subject by administering to the subject a composition comprising G-CSF, plerifaxor, or a combination of G-CSF and plerifaxor;
(b) enriching the PBMCs for CD34+ cells by magnetic separation to generate a population of CD34-enriched cells; and
(c) contacting the CD34-enriched cells with a lentiviral particle comprising a recombinant lentiviral genome, comprising in 5′ to 3′ order:
(i) a lentiviral 5′ long terminal repeat (LTR);
(ii) the expression cassette of any one of claims 21 to 30; and
(iii) a lentiviral 3′LTR,
wherein the recombinant lentiviral genome is replication incompetent.

47. A method of treating infantile malignant osteopetrosis (IMO) in a subject having or suspected of having IMO, comprising administering the modified cell of any one of claims 35 to 44 or the pharmaceutical composition of claim 45 to the subject.

48. The method of claim 47, wherein the method repopulates the HSC niche with modified cells expressing TCIRG1 or a functional variant thereof.

49. The method of claim 47 or 48, wherein the method repopulates the osteoclast niche with modified cells expressing TCIRG1 or a functional variant thereof.

50. The method of any one of claims 47 to 49, wherein the method treats, ameliorates, prevents, reduces, inhibits, or relieves IMO.

51. The method of any one of claims 47 to 50, wherein the method extends the mean overall survival of treated subjects by at least 1, 2, 3, 4, 5, 6, 7, 8, 9, 10 or more years.

52. The method of any one of claims 47 to 51, wherein the method prevents the death of the subject from IMO.

53. The method of any one of claims 47 to 52, wherein the subject is a human.

54. The method of any one of claims 47 to 53, wherein the subject exhibited symptoms of IMO before treatment.

55. The method of any one of claims 47 to 54, wherein the subject was identified as having reduced or non-detectable expression of TCIRG1 before treatment.

56. The method of any one of claims 47 to 55, wherein the subject was identified as having a mutated TCIRG1 gene.

57. The method of any one of claims 47 to 56, wherein the subject is an infant.

58. The method of any one of claims 47 to 57, wherein the method comprises autologous treatment.

59. The method of any one of claims 47 to 58, wherein administration is performed via a intravenous infusion.

60. A recombinant lentiviral genome for use in the preparation of a medicament for treating or preventing infantile malignant osteopetrosis (IMO), wherein the lentiviral genome comprises in 5′ to 3′ order:
(i) a lentiviral 5′ long terminal repeat (LTR),
(ii) the expression cassette of any one of claims 21 to 30, and
(iii) a lentiviral 3′LTR; and
wherein the recombinant lentiviral genome is replication incompetent.

61. A lentiviral particle for use in the preparation of a medicament for treating or preventing infantile malignant osteopetrosis (IMO), comprising a recombinant lentiviral genome,
wherein the lentiviral genome comprises in 5′ to 3′ order:
(i) a lentiviral 5′ long terminal repeat (LTR),
(ii) the expression cassette of any one of claims 21 to 50, and
(iii) a lentiviral 3′LTR; and
wherein the recombinant lentiviral genome is replication incompetent.

62. A transfer plasmid comprising the expression cassette of any one of claims 24-30.

63. The transfer plasmid of claim 62, further comprising an RNA-OUT sequence.

64. The transfer plasmid of claim 63, wherein the RNA-OUT sequence is SEQ ID NO: 22.

65. The transfer plasmid of claim 62 or claim 63, wherein the RNA-OUT sequence is configured such that the transfer plasmid is capable of stable propagation in a packaging cell line.

66. A method of producing a lentiviral particle, comprising transforming a bacterial cell with the transfer plasmid of any one of claims 1-19 or claims 62-65, such that the transfer plasmid is replicated, isolating the replicated transfer plasmid, and transducing a packaging cell line with the replicated transfer plasmid, and optionally one or more additional plasmids, thereby producing the lentiviral particle.

67. A method of producing a lentiviral particle, comprising transfecting a packaging cell line with the transfer plasmid of any one of claims 1-19 or claims 62-65, and optionally one or more additional plasmids, and culturing said packaging cell line.

68. The method of claim 67, wherein the transfer plasmid is stably propagated.

69. The method of claim 68, wherein the transfer plasmid is stably propagated in a bacterial host at 30-37° C. using shake flasks or fermentation for at least 1, 2, 3, 4, 5, 6, or 7 days.

70. A lentiviral particle produced according to a method of any one of claims 66-69.
